(19) United States
(12) Patent Application Publication
(10) Pub. No.: US 2025/0000876 A1
(43) Pub. Date: Jan. 2, 2025

(54) Novel therapeutic agent for inflammatory disease

(71) Applicants: Gachon University of Industry-Academic cooperation Foundation, Gyeonggi-do (KR); Eulji University Industry Academy Cooperation Foundation, Gyeonggi-do (KR); Kyungpook National University Industry-Academic Cooperation Foundation, Daegu (KR)

(72) Inventors: Dongwoo KHANG, Seoul (KR); Jun-Young PARK, Incheon (KR); Youn Joo KANG, Seoul (KR); Sang-Hyun KIM, Daegu (KR); Song KWON, Incheon (KR)

(21) Appl. No.: 18/748,247

(22) Filed: Jun. 20, 2024

Related U.S. Application Data
(62) Division of application No. 17/389,449, filed on Jul. 30, 2021, now abandoned.

(30) Foreign Application Priority Data
Jul. 30, 2020 (KR) 10-2020-0095406

(51) Int. Cl. A61K 31/573; A61K 47/69; A61P 29/00; B82Y 5/00

(52) U.S. Cl. CPC A61K 31/573; A61K 47/6933; A61P 29/00; B82Y 5/00

(57) ABSTRACT

The present invention provides a novel therapeutic agent for treating inflammatory diseases which reduces the pro-inflammatory response and increases M2 macrophages according to the repolarization of macrophages, thereby exhibiting fewer side effects and excellent anti-inflammatory effects even at low doses compared to conventional steroid anti-inflammatory agent.

### BACKGROUND TO RELATED APPLICATION

This application is a divisional of U.S. application Ser. No. 17/389,449, filed Jul. 30, 2021, which claims the benefit of Republic of Korea Application No. 10-2020-0095406, filed Jul. 30, 2020, the contents of which are incorporated herein by reference.

### RELATED APPLICATION

This application claims the benefit of priority to Korean Patent Application No. 10-2020-0095406, filed on Jul. 30, 2020. The contents of the above-described application are incorporated herein in their entirety.

### REFERENCE TO AN ELECTRONIC SEQUENCE LISTING

The contents of the electronic sequence listing (33010.026US2_sequence listing.xml; Size: 46,168 bytes; and Date of Creation: Sep. 11, 2024) is herein incorporated by reference in its entirety.

### TECHNICAL FIELD

The present invention relates to a novel therapeutic agent for inflammatory diseases, and more particularly, to a novel therapeutic agent for inflammatory diseases that exhibits high anti-inflammatory effects while having few side effects.

### BACKGROUND

Currently used therapeutics for treating arthritis are administered by the oral or intra-articular route, and include five major drugs, including corticosteroids, analgesics, nonsteroidal anti-inflammatory drugs, and abiotic and biological disease-modifying anti-rheumatic drugs. Among them, corticosteroids are very strong anti-inflammatory agents and have been used for symptomatic anti-arthritis agent for about 60 years by intra-articular injection. Many studies have been reported on the use of intra-articular corticosteroid injections in arthritis, and the evidence is that it is currently widely used in rheumatoid arthritis (RA) and osteoarthritis (OA) (E. Maheu, et al., Semin. Arthritis Rheum., 48 (4): 563-572. 2019). In particular, triamcinolone, a synthetic glucocorticoid, has been widely used as an intra-articular injection for the treatment of arthritis, but in previous studies, it was reported that intra-articular injection of triamcinolone generally causes secondary adrenal insufficiency (J. Paik, et al., Drugs, 79 (4): 455-462, 2019). In addition, repeated long-term administration and high-dose intra-articular corticosteroid injection eventually induce drug resistance, hyperglycemia in diabetic patients, Cushing syndrome, steroid-like arthroplasty, osteoporosis, and skeletal fractures and serious local and systemic side effects (in a dose dependent manner) including gastrointestinal bleeding.

On the other hand, RA and OA are diseases that cause inflammation in the joints, and OA is very common in adults over 50 years old. It is characterized by persistent inflammation and the formation of hyperplastic and invasive synovial membranes in the joints. Synoviocytes (fibroblast-like synoviocytes, FLSs) are important stromal cells of the joints that contribute to the pathogenesis of arthritis (B. Bartok et al., Immunol. Rev., 233 (1): 233-255, 2010). The invasive phenotype of the FLSs promotes the degradation and destruction of cartilage through the production of matrix metalloproteinases (MMPs), pro-inflammatory cytokines and chemokines (A. Mor, S. B. et al, Clin. Immunol., 115 (2): 118-128, 2005). Recently, macrophages have begun to be considered critically involved in the pathogenesis of arthritis (R. W. Kinne et al., Arthritis Res., 2 (3): 189-202, 2000). Macrophages represent a classical pro-inflammatory (M1) phenotype and an anti-inflammatory (M2) phenotype. The M1 macrophages dominate in the early stages of inflammation and induce inflammation by secreting pro-inflammatory cytokines such as interleukin (IL)-1β and tumor necrosis factor (TNF)-α. In contrast, M2 macrophages secrete anti-inflammatory cytokines and are implicated in the relief of inflammation (S. Behzadi et al., Chem Soc Rev, 46 (14), 4218-4244. 2017). Macrophages in RA joints are predominantly M1 phenotype and promote RA progression by releasing various inflammatory cytokines. Therefore, macrophages have emerged as potential targets for the treatment of inflammatory diseases (H. Hillaireau et al., Cell Mol. Life Sci., 66 (17): 2873-2896. 2009).

### SUMMARY OF THE INVENTION

### Technical Problem

However, detailed studies related to the crosstalk between FLSs and macrophages in arthritis and the increase in the anti-inflammatory effect of nano-drugs have not yet been clearly elucidated.

The present invention is derived to solve various problems including the above problems, and an object of the present invention is to provide a novel therapeutic agent for inflammatory diseases that has few side effects and exhibits a high anti-inflammatory effect even at a low dose. However, these problems are exemplary, and the scope of the present invention is not limited thereto.

### Technical Solutions

According to one aspect of the present invention, there is provided a therapeutic agent for treating inflammatory disease comprising gold nanoparticles (AuNPs)-steroid complex as an effective ingredient, wherein the gold nanoparticles are coated with PEG and a steroid anti-inflammatory agent is loaded to the surface of the gold nanoparticle via covalent or non-covalent bond.

According to another aspect of the present invention, there is provided a method of treating inflammatory disease in a subject comprising administrating gold nanoparticles (AuNPs)-steroid complex to the subject, wherein the gold nanoparticles are coated with PEG and a steroid anti-inflammatory agent is loaded to the surface of the gold nanoparticle via covalent or non-covalent bond.

According to another aspect of the present invention, there is provided use of a gold nanoparticle-steroid complex in the manufacture of medicament for treating inflammatory disease loaded with a steroid-based anti-inflammatory agent on the surface by covalent or non-covalent bonding on gold nanoparticles (AuNPs) coated with PEG on the surface as an active ingredient.

### Effect of the Invention

The novel therapeutic agent for treating inflammatory diseases according to the present invention as described above reduces the pro-inflammatory response and increases M2 macrophages according to the repolarization of macrophages. Therefore, the present invention may be used as a new drug candidate for the treatment of autoimmune diseases such as arthritis or atopy. Of course, the scope of the present invention is not limited by these effects.

### BRIEF DESCRIPTION OF DRAWINGS

FIG. 1A is a schematic diagram representing the chemical structure of Triam-AuNPs according to an embodiment of the present invention.

FIG. 1B is a TEM image of a Triam-AuNP according to an embodiment of the present invention showing the distribution of Au (yellow), S (a component of PEG; orange) and F (a component of Triamcinolone; red) measured using EDS mapping. Scale bar represents 20 nm.

FIG. 1C is a graph showing the results of analyzing the hydrodynamic size distribution of the Triam-AuNP complex, citrate NPs, pegylated NPs, and Triamcinolone according to an embodiment. The size of NPs increased by 15 nm after conjugation with Triamcinolone.

FIG. 1D is a graph showing the results of analysis of average hydrodynamic sizes and electrical potentials of citrate NPs, pegylated NPs, Triam, and Triam-AuNPs according to an embodiment.

FIG. 1E is a graph showing the results of analyzing the UV-vis spectrometry results showing the Triamcinolone peak at 242 nm identifying Triam-AuNPs according to an embodiment of the present invention.

FIG. 1F is a graph showing the results of measuring the calibration curve of AuNPs.

FIG. 1G is a graph showing the results of measuring the calibration curve of Triamcinolone.

FIG. 1H is a graph showing the results of analyzing the FT-IR peaks of PEG, AuNPs and Triam-AuNPs according to an embodiment of the present invention. The same peaks were observed for Triam and Traim-AuNPs.

FIG. 2A is a confocal microscopic image showing TNF-α-stimulated FLSs treated with nanodrugs, which is drawn to an analysis of FLSs and macrophages uptakes. TNF-α-stimulated FLSs and LPS-stimulated macrophages treated with different types of uptake inhibitors were analyzed. Caveolae-mediated endocytosis has been identified as the major pathway used by FLSs for uptake of applied nanodrugs.

FIG. 2B is a confocal microscopic image showing LPS-stimulated macrophages treated with nanodrugs, which is drawn to an analysis of FLSs and macrophage uptakes. LPS-stimulated macrophages treated with different types of uptake inhibitors were analyzed (major: micropinocytosis and clathrin-mediated endocytosis, minor: caveolae-mediated endocytosis).

FIG. 2C is a graph showing the results of analyzing the fluorescence intensity (absorption) of TNF-α-stimulated FLSs treated with nanodrugs related to FLSs and macrophage uptake assay.

FIG. 2D is a graph showing the results of analyzing the fluorescence intensity (absorption) of LPS-stimulated macrophages treated with nanodrugs related to FLSs and macrophage uptake analysis. The data are presented as mean+SEM (n=10). *, ** and *** correspond to *p<0.05, ** p<0.01 and ***p<0.001, respectively, compared to the indicated groups.

FIG. 3A is a series of confocal microscopic images of TNF-α-stimulated FLSs representing ROS analysis for FLSs and macrophages. Triam-AuNPs according to an embodiment of the present invention removed the intracellular ROS higher than AuNPs and Triam. Scale bar represents 150 μm.

FIG. 3B is a series of confocal microscopic images LPS-stimulated macrophages representing ROS assay for FLSs and macrophages. Triam-AuNPs according to an embodiment of the present invention removed the intracellular ROS higher than AuNPs and Triam. Scale bar represents 150 μm.

FIG. 3C is a set of graphs showing the results of analyzing the gene expression of iNOS representing ROS analysis for FLSs (right) and M1 macrophages (left). Gene expression of iNOS directly coupled with ROS generation was suppressed by Triam-AuNPs in active FLSs and M1 macrophages.

FIG. 4A is a series of graphs showing analyses of expression of pro-inflammatory cytokines (TNF-α, IL-1β, IL-6, MMP-1 and MMP-3) in FLSs of RA and OA patients representing the therapeutic efficacy of Triam-AuNPs according to an embodiment of the present invention at a low dose compared to a high dose Triam.

FIG. 4B is a series of graphs showing analyses of expression of pro-inflammatory mRNA (TNF-α, IL-1β, IL-6, MMP-1 and MMP-3) in FLSs of RA and OA patients representing the therapeutic efficacy of low-dose Triam-AuNPs according to an embodiment of the present invention in comparison with high-dose Triam. These data suggest that Triam-AuNP treatment inhibited more pro-inflammatory cytokine release than Triam treatment in FLSs of OA and RA patients. The data are presented as mean+SEM (n=3). *, ** and *** correspond to *p<0.05, ** p<0.01 and ***p<0.001, respectively, compared to the indicated groups.

FIG. 5A is a series of graphs showing the results of analyzing the down-regulation of pro-inflammatory cytokines (TNF-α, IL-1β and IL-6) expression in FLSs by Triam and Triam-AuNPs treatment according to an embodiment of the present invention.

FIG. 5B is a series of graphs showing the results of analyzing the expression of anti-inflammatory cytokines (IL-4, IL-10 and Arg-1) in FLSs treated with Triam and Triam-AuNPs according to an embodiment of the present invention. Triam and Triam-AuNPs both affect pro-inflammatory cytokines, but only Triam-AuNPs upregulate anti-inflammatory cytokines (IL-4 and Arg-1). Triam did not induce anti-inflammatory cytokine release by FLSs. However, an increased anti-inflammatory response was observed after Triam-AuNP treatment. Data are presented as mean+SEM (n=3). *, ** and *** correspond to *p<0.05, **p<0.01 and ***p<0.001, respectively, compared to the indicated groups.

FIG. 5C is a schematic diagram showing the activation of FLSs by M1 macrophages and the inactivation of activated FLSs by Triam and Triam-AuNPs.

FIG. 6A is a series of graphs showing analyses macrophage repolarization by nanodrugs according to an embodiment of the present invention. Activated FLSs differentiated macrophages into M1 macrophages.

FIG. 6B is a series of graph showing the results of examining macrophage repolarization by a nanodrug according to an embodiment of the present invention. By treatment with Triam-AuNPs, anti-inflammatory cytokine gene expression in M2 macrophages was significantly increased. Unlike Triam-AuNPs, Triam did not induce repolarization of M1 macrophages to M2 macrophages.

FIG. 6C is a schematic diagram showing macrophage activation by FLSs and inactivation of activated M1 macrophages by Triam and Triam-AuNPs according to an embodiment of the present invention. An increased anti-inflammatory response (repolarization) was observed after Triam-AuNP treatment. Data are presented as mean+SEM (n=3). *, ** and *** correspond to *p<0.05, **p<0.01 and ***p<0.001, respectively, compared to the indicated groups.

FIG. 7A is a schematic diagram showing an experimental schedule for injection of nanodrugs and CIA modeling according to an embodiment of the present invention.

FIG. 7B is a series of representative photographs on CIA model mice administrated with saline, AuNPs, Triam (2 mg/kg and 5 mg/kg) and Triam-AuNPs (2 mg/kg and 5 mg/kg) into the ankle joint. The mice were sacrificed and tissues were subjected with H&E staining and safranin-O staining in order to investigate the treatment effect of nanodrugs.

FIG. 7C is a graph showing Paw score of DBA/1 mice administrated with saline, AuNPs, Triam (2 mg/kg and 5 mg/kg) and Triam-AuNPs (2 mg/kg and 5 mg/kg) into the ankle joint during CIA development up to 48 days representing the therapeutic effect of the nanodrugs according to an embodiment of the present invention.

FIG. 7D is a graph showing the results of analyzing cartilage content DBA/1 mice administrated with saline, AuNPs, Triam (2 mg/kg and 5 mg/kg) and Triam-AuNPs (2 mg/kg and 5 mg/kg) into the ankle joint during CIA development up to 48 days representing the therapeutic effect of the nanodrugs according to an embodiment of the present invention. Data are presented as mean±SEM (n=3). *, ** and *** correspond to *p<0.05, *p<0.01 and ***p<0.001, respectively, compared to the indicated groups.

FIG. 8A is a series of immunofluorescent staining confocal images for IL-6, IL-1, IFN-γ and TNF-α in one ankle joint of CIA model mice, treated with saline, AuNP, Triam (2 mg/kg and 5 mg/kg) and Triam-AuNPs (2 mg/kg and 5 mg/kg) for 48 days. All pro-inflammatory cytokines were significantly inhibited in the high-dose Triam-AuNP group (5 mg/kg). Scale bar represents 50 μm.

FIG. 8B is a series of graphs showing the results of quantitative analysis of immunofluorescence for IL-6, IL-1, IFN-γ and TNF-α by analyzing the immunofluorescence of CIA model mice. Overall, the high-dose Triam-AuNP (5 mg/kg) outperformed the anti-inflammatory efficacy of the high-dose Triam (5 mg/kg). Data are presented as mean±SEM (n=3). *, ** and *** correspond to *p<0.05, **p<0.01 and ***p<0.001, respectively, compared to the indicated groups.

FIG. 9A is a series of immunofluorescence confocal images confirming CD86 (M1 marker) and Dectin-1 (M2 marker) in ankle joints treated with various drugs to investigate the induction of macrophage repolarization in the synovial membrane by nanodrugs.

FIG. 9B is a series of graphs showing the results of analyzing the strength of CD86 (M1 marker) and Dectin-1 (M2 marker) in the ankle joint treated with various drugs to investigate the induction of macrophage repolarization in the synovial membrane by the nanodrug. Triam-AuNPs effectively modulated macrophage repolarization in inflamed synovial membranes. Scale bars indicate 6000 μm (black) and 850 μm (red). Data are presented as mean±SEM (n=3). *, ** and *** correspond to *p<0.05, **p<0.01 and ***p<0.001, respectively, compared to the indicated groups.

FIG. 10 is a schematic diagram showing the inflammatory crosstalk between FLSs and macrophages. FLSs and macrophages were stimulated and inactivated with each other. Reduced inflammatory macrophage responses (mediated by M1 macrophages) and M2 differentiation provide favorable conditions in the inflammatory synovial membrane through inactivation of the inflammatory response of FLSs.

FIG. 11A is a graph showing the analysis result of the polydispersity index treated with various drugs including Triam-AuNPs according to an embodiment of the present invention.

FIG. 11B is a graph showing the results of analyzing the fluorescence intensity according to AuNP citrate and Triam-Alexa 488 AuNPs treatment according to an embodiment of the present invention.

FIG. 12A is a graph showing the results of analyzing the H₂O₂ concentration when treated with various drugs including Triam-AuNPs according to an embodiment of the present invention.

FIG. 12B is a photograph showing the results of observing O₂ production when treated with various drugs including Triam-AuNPs according to an embodiment of the present invention.

FIG. 13A is a series of graphs showing the results of analyzing the pro-inflammatory characteristics of RA patient-derived primary FLSs representing the anti-inflammatory effect of Triam-AuNPs treatment according to an embodiment of the present invention.

FIG. 13B is a series of graphs showing the results of analyzing the pro-inflammatory characteristics of OA patient-derived primary FLSs representing the anti-inflammatory effect of Triam-AuNPs treatment according to an embodiment of the present invention.

FIG. 14A is a series of graphs showing the results of analyzing the levels of inflammatory cytokines (TNF-α, IL-1β, IL-6, and iNOS) representing the anti-inflammatory effect of Triam-AuNPs treatment according to an embodiment of the present invention.

FIG. 14B is a graph showing the results of analyzing the anti-inflammatory cytokines (IL-4, IL-10 and Arg-1) levels representing the anti-inflammatory effect of Triam-AuNPs treatment according to an embodiment of the present invention.

FIG. 15 is an immunostaining photograph of CD248 showing the inflammatory phenotype that exacerbates joint damage in RA representing the anti-inflammatory effect of Triam-AuNPs treatment according to an embodiment of the present invention.

FIG. 16A is a schematic diagram schematically showing the chemical structure of Dex-AuNPs according to an embodiment of the present invention.

FIG. 16B is a TEM image of Dex-AuNPs according to an embodiment of the present invention showing the distribution of Au (green), S (a component of PEG; orange), and F (a component of Dexamethasone: red) measured using EDS mapping. Scale bar represents 100 nm.

FIG. 16C is a graph showing the results of analyzing the hydrodynamic size distribution of the Dex-AuNPs complex, AuNPs, pegylated NRs and DEX according to an embodiment of the present invention, which increased by 10 nm in size after conjugation with DEX.

FIG. 16D is a graph showing the results of analyzing the average hydrodynamic sizes and electrical potentials of AuNPs, pegylated NRs, DEX, and Dex-AuNPs according to an embodiment of the present invention.

FIG. 16E is a graph showing the UV-vis results showing the DEX peak at 242 nm identified in Dex-AuNPs according to an embodiment of the present invention.

FIG. 17A is a result of superficial analysis of symptom relief of atopic dermatitis by applying Dex-AuNPs after induction of atopic dermatitis (upper) and histopathological microscopic images (lower).

FIG. 17B is a graph analyzing the results of skin thickness relief among symptoms of atopic dermatitis by applying Dex-AuNPs after induction of atopic dermatitis.

FIG. 17C is a series of graphs analyzing the results of confirming the thickness change of atopic dermatitis by applying Dex-AuNPs after induction of atopic dermatitis with a microscopic microscope, and measuring serum immunoglobulin and cytokine expression results in atopic dermatitis tissues among immunological symptoms of atopic dermatitis.

FIG. 18A is a series of photographs showing the surface area and histopathological results by treating Dex-AuNPs after inducing psoriatic dermatitis using Aldara cream (5% Imiquimod).

FIG. 18B is a series of graphs analyzing serum level of immunoglobulin IgG2a (upper) and MPO (myeloperoxidase, lower) when treated with various drugs including Dex-AuNPs after induction of psoriatic dermatitis.

FIG. 18C is a series of graphs showing the result of analyzing skin thickness, scaling, erythema, and skin thickness increase by treatment with Dex-AuNPs after inducing psoriatic dermatitis, representing skin score.

### BETS MODES

### Definition of Terms

As used herein, the term “crosstalk” refers to the case in which one or more components in one signal transduction pathway affect another. Biological interactions can be achieved in a number of ways, with interactions between signaling cascade proteins being the most common. In these signal transduction pathways, there are often shared components that can interact with either pathway. More complex interactions can be observed with crossmembrane crosstalk between the extracellular matrix (ECM) and the cytoskeleton.

As used herein, the term “triamcinolone” is a white crystalline powder-like adrenocortical steroid, used as an anti-inflammatory agent.

As used herein, the term “dexamethasone” is a synthetic adrenocortical hormone with anti-inflammatory action, and prepared as tablets, injections, ophthalmic preparations, oral ointments, nasal sprays, creams, etc.

As used herein, the term “matrix metalloproteinases (MMP)” is a proteolytic enzyme that degrades the matrix components of bone and cartilage and plays a major role in pathological conditions such as atherosclerosis, tumor invasion and arthritis. Among them, MMP-1 and MMP-3 are expressed in regions where rheumatoid arthritis (RA)-related bone erosion occurs, and are known as predictors of joint destruction in early RA patients.

As used herein, the term “PEGylation” refers to a process of covalently and non-covalently attaching polyethylene glycol, a non-toxic additive, to increase half-life of drugs.

### DETAILED DESCRIPTION OF THE INVENTION

According to one aspect of the present invention, there is provided is a pharmaceutical composition for treating inflammatory disease comprising a gold nanoparticle-steroid complex in which a steroid anti-inflammatory agent is loaded on the surface of gold nanoparticle (AuNPs) coated with PEG by covalent or non-covalent bond as an active ingredient.

In the pharmaceutical composition, the gold nanoparticles may be gold nanoparticles whose surface is thiolated by a thiol-functionalized compound, and the thiol-functionalized compound may be cystamine, thiol-PEG, glutathione, cysteine, or mercaptopropionate.

In the pharmaceutical composition, the steroid anti-inflammatory agent may be triamcinolone, hydrocortisone, prednisolone, betamethasone, or dexamethasone.

In the pharmaceutical composition, the inflammatory disease may be arthritis, psoriasis or atopic dermatitis, and the arthritis may be osteoarthritis or rheumatoid arthritis.

In the pharmaceutical composition, the PEG may have a number average molecular weight of 3000 Da to 5000 Da.

In the pharmaceutical composition, the loading ratio of the steroid anti-inflammatory agent to the gold nanoparticles coated with PEG may be 6 to 4 to 7 to 3, and the size of the gold nanoparticles coated with PEG may be 20 to 100 nm. And the steroid-based anti-inflammatory agent may be covalently bonded to the thiol-functionalized gold nanoparticles.

Pharmaceutical composition may reduce the expression of pro-inflammatory cytokines and increase the expression of anti-inflammatory cytokines, and the pro-inflammatory cytokines are TNF-α, IL-1β, IL-6, INF-γ, MMP-1 or MMP-3 and the anti-inflammatory cytokine may be IL-4, IL-10 or Arg-1 and the anti-inflammatory cytokine may induce repolarization from M1 macrophages to M2 macrophages.

According to another aspect of the present invention, there is provided is a method of treating inflammatory disease in a subject comprising administrating therapeutically effective amount of a gold nanoparticle-steroid complex in which a steroid anti-inflammatory agent is loaded on the surface of gold nanoparticle (AuNPs) coated with PEG by covalent or non-covalent bond to the subject.

According to another aspect of the present invention, there is provided is use of a gold nanoparticle-steroid complex in which a steroid anti-inflammatory agent is loaded on the surface of gold nanoparticle (AuNPs) coated with PEG by covalent or non-covalent bond in the manufacture of a therapeutic agent for treating inflammatory disease.

In the method, the pharmaceutical composition may be administered through oral or parenteral administration, and oral administration is more preferable, but is not limited thereto. When administered parenterally, it is possible to administer it through various routes, such as intravenous injection, intranasal inhalation, intramuscular administration, intraperitoneal administration, and transdermal absorption.

The preferred dosage of the pharmaceutical composition of the present invention varies depending on the condition and body weight, the severity of the disease, the drug form, the route and duration of administration, but may be appropriately selected by those skilled in the art. Preferably, it is administered at 0.01 to 1000 mg/kg per day for adults (60 kg body weight), and to be more effective, it is preferably administered at 1 to 100 mg/kg. The dosage and frequency of administration may be administered once a day, or may be administered in several divided doses. The dosage and frequency of administration do not limit the scope of the present invention in any way.

The pharmaceutical composition according to an embodiment of the present invention may include a pharmaceutically acceptable carrier, and may additionally include a pharmaceutically acceptable adjuvant, excipient or diluent in addition to the carrier.

As used herein, the term “pharmaceutically acceptable” refers to components which are physiologically acceptable and does not normally cause gastrointestinal disorders, allergic reactions such as dizziness or similar reactions when administered to humans. Examples of such carriers, excipients and diluents include lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oil. In addition, fillers, anti-agglomeration agents, lubricants, wetting agents, fragrances, emulsifiers and preservatives may be further included. In addition, the pharmaceutical composition according to an embodiment of the present invention may be formulated using a method known in the art to enable rapid, sustained or delayed release of the active ingredient when administered to a mammal. Formulations include powders, granules, tablets, emulsions, syrups, aerosols, soft or hard gelatin capsules, sterile injectable solutions, and sterile powder forms.

Recently, studies on gold nanoparticles (AuNPs)-based drug delivery have been conducted to improve the treatment of inflammatory diseases such as arthritis, and modulation of the immune response using nanomaterials is a promising strategy for the treatment of arthritis diseases (H. Hillaireau, et al., Cell Mol. Life Sci., 66 (17): 2873-96. 2009). The biomedical applications of AuNPs are rapidly expanding due to their diverse properties, including relatively low toxicity, high capacity for cell targeting, easily functionalized surfaces, sustained drug release, and the tunable light absorption and heat treatment capabilities of these nanoparticles (NPs). Although most nanomaterials are recognized as unsuitable for clinical application due to side effects and clearance problems during intravenous injection, AuNPs are still considered promising biomaterials in rheumatological applications (Wang et al., Biomater. Sci., 5 (8): 1407-1420. 2017).

In addition, understanding the crosstalk between synoviocytes and macrophages is very important for developing strategies for regulating the inflammatory response in inflamed synovium. Simultaneous modulation of pro- and anti-inflammatory responses of synovial cells and macrophages (repolarization) is important in the treatment of arthritis.

In the present invention, pegylated AuNPs with a size of 20 nm were conjugated with clinical grade Triamcinolone (hereinafter, referred to as “Triam”). The AuNPs conjugated with triamcinolone (hereinafter, referred to as “Triam-AuNPs”) effectively inhibited inflammation mediated by patient-derived primary RA FLSs and patient-derived OA FLSs with the same aspect. In addition, Triam-AuNPs simultaneously increased the anti-inflammatory response of FLSs and macrophages repolarized from M1 (pro-inflammatory) to M2 (anti-inflammatory) phenotype. In an in vivo experiment, macrophages were repolarized from M1 to M2 phenotype or only the pro-inflammatory response of FLS and M1 macrophages was inhibited without activation of the anti-inflammatory response of FLSs. Modulation of dynamic immunomodulation (both down-regulation of pro-inflammatory responses and up-regulation of anti-inflammatory responses) with Triam-AuNPs may provide ideal conditions for the treatment of arthritis models. In addition, the present inventors demonstrated that AuNPs themselves can catalyze the degradation of H₂O₂, an essential element for ROS production, but despite the strong H₂O₂ degradation ability of AuNPs, the effect of AuNPs on intracellular ROS levels was not significant. In contrast, Triam-AuNPs not only reduced ROS and inflammatory levels of TNF-α-stimulated FLS, but also exhibited the ability to clear ROS from activated macrophages.

In addition, in experiments on a collagen-induced arthritis (CIA) mouse model, compared with Triam alone treatment, Triam-AuNPs showed significant pro-inflammatory markers (IL-6, IL-1, INF-γ, and TNF-α) in cartilage tissue and increased the number of M2 macrophages in the cartilage tissue by effectively repolarizing the macrophage activity in the inflammatory synovial membrane, thereby greatly promoting cartilage regeneration (indicated by Safranin-O staining). The above results clearly suggest that the Triam-AuNPs of the present invention have immune crosstalk between FLSs and macrophages in inflammatory joints.

In the present invention, it was found that the interaction between synovial cells and macrophages is significantly involved in the activation and inactivation of the inflammatory response in the synovial membrane. In addition, according to the increase in the secretion of anti-inflammatory cytokines by FLSs and macrophages induced by the treatment of Triam-AuNPs, the nanodrugs of the present invention (Triam-AuNPs) more strongly modulate the immune response than conventional glucocorticoid (GC) drugs. Therefore, the nanodrug of the present invention, Triam-AuNPs, can be used as a next-generation novel drug candidate for the treatment of RA and OA.

Hereinafter, the present invention will be described in more detail through examples. However, the present invention is not limited to the embodiments disclosed below, but it is provided to fully inform the methods be implemented in a variety of different forms.

### General Methods

### Synthesis of AuNPs

Citrate-stabilized AuNPs (20 nm) of the present invention were synthesized according to the traditional Turkevich-Frens method. Specifically, after adding a 2.2 mM sodium citrate solution to distilled water (DW), it was heated with a heating mantle under vigorous stirring for 15 minutes. When the temperature of the solution reached 100° C., a gold (III) chloride trihydrate (HAuCl₄) solution (25 mM, Sigma, St. Louis, MO, USA; Cat: 5209258) was added to obtain a gold seed solution. After synthesizing the gold seed, the temperature of the solution was lowered to 90° C., HAuCl₄ solution (25 mM) was added, and the reaction solution was vigorously stirred for 30 minutes. Thereafter HAuCl₄ solution (25 mM) was added and the process of stirring for 30 minutes was repeated twice to obtain 20 nm AuNPs coated with citrate ions, which are negatively charged.

### Conjugation of Triam and AuNPs

Conjugation of Triam-AuNPs was prepared by first coating citrate-stabilized AuNPs with α-mercapto-ω-carboxy PEG solution (10 μM, MW=5 kDa, Sigma; Cat No.: 712523) under moderate agitation overnight. At the same time, Triam solution was prepared by mixing Triamcinolone acetonide (10 mg/mL) and carbonyldiimidazole (10 mg) in 1 mL of dimethylformamide (DMF) for 2 hours, then adding 1 mL of cysteamine (20 μM) and reacting overnight. The PEGylated AuNP solution was then centrifuged (4,000 rpm, room temperature, 15 min, Fleta 5, Hanil, Korea) using a filter tube (Ultracell®-30K, 30 kDa, Millipore, Billerica, MA, USA; Cat: UFC903024) and washed three times with DW. Then, the pegylated AuNP solution was resuspended in 4 mL of DW and added to the Triam solution for conjugation at 4° C. for 24 hours. Finally, the Triam-AuNPs solution was collected after washing with DW by centrifugation at 4,000 rpm for 15 minutes at 4° C., and the Triam-AuNPs were resuspended in 1 mL of DW and stored at 4° C. for use.

Conjugation of Dexamethasone with AuNPs

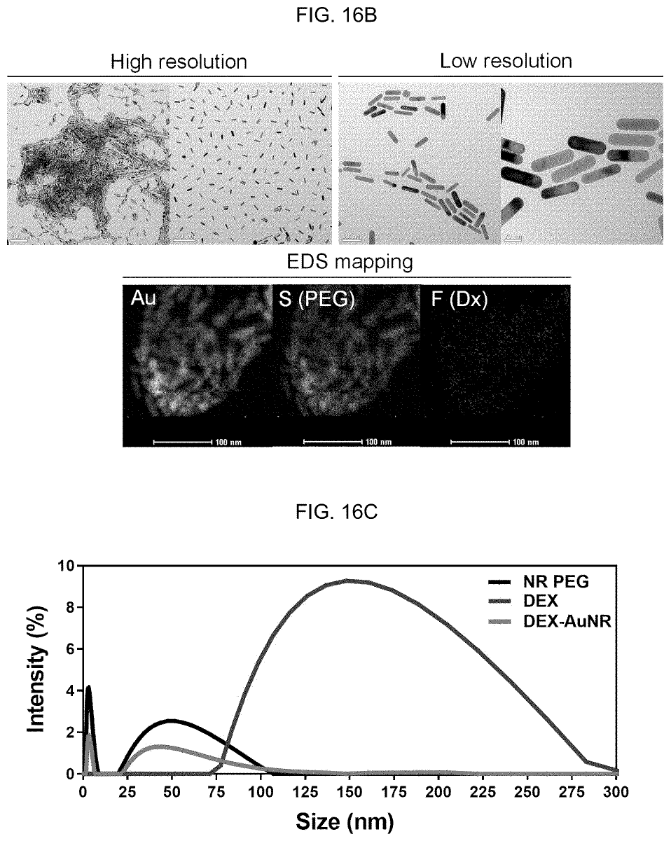

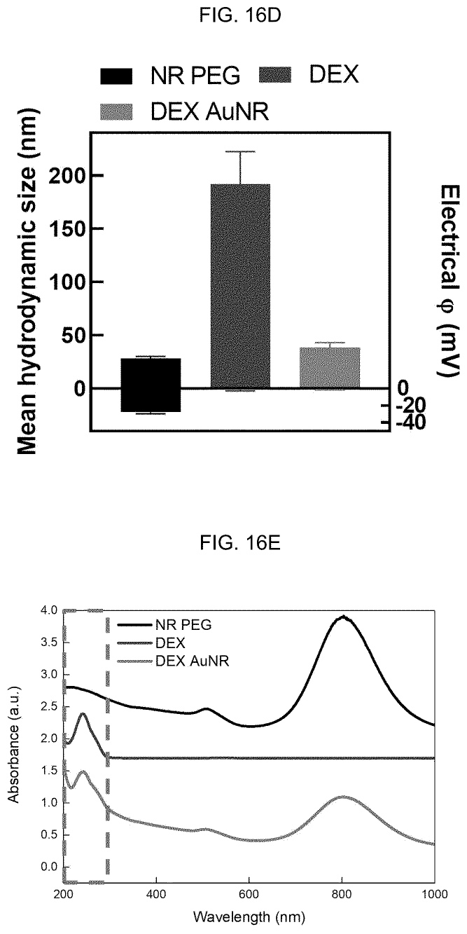

Conjugation of Dex-AuNPs was performed by first coating AuNPs stabilized with cetrimonium bromide with α-mercapto-ω-carboxy PEG solution (10 μM, MW=5 kDa, Sigma; Cat No.: 712523) under moderate stirring overnight (FIG. 16A). At the same time, dexamethasone (10 mg) and carbonyldiimidazole (10 mg) were mixed in 1 mL of dimethylformamide (DMF) for 2 hours, then 1 mL of cysteamine (20 μM) was added and reacted overnight. The PEGylated AuNPs solution was then centrifuged (4,000 rpm, room temperature, 15 min, Fleta 5, Hanil, Korea) using a filter tube (Ultracell®-30K, 30 kDa, Millipore, Billerica, MA, USA; Cat: UFC903024) and washed three times with DW. Then, the pegylated AuNPs solution was resuspended in 4 mL of DW and added to the Dex solution for conjugation at 4° C. for 24 hours. Finally, the Dex-AuNPs solution was collected after washing with DW by centrifugation at 4,000 rpm for 15 minutes at 4° C., and the Dex-AuNPs were obtained by resuspending in 1 mL of DW and stored at 4° C. for use.

### Analyses of Physicochemical Properties

The concentration of Triam covalently bound to AuNPs and the concentration of AuNPs were analyzed through absorbance measurement using a UV-vis spectrophotometer (Libra S50, Biochrom, Cambridge, UK). Specifically, the concentration of AuNPs was quantified by measuring the peak absorbance at 520 nm, and the concentration of Triam on AuNPs was determined by subtracting this value from the initial and final absorbances of the conjugated AuNPs and measuring the absorbance at 242 nm. The concentration of each solution was inferred based on a linear standard curve for AuNPs. The size distribution and potential of the Triam-AuNPs complex were analyzed using a Zetasizer Nano (Malvern, UK; Cat: ZS90), and for FTIR (Nicolet iS5, Thermo, MA, Waltham, USA) analysis, samples were completely lyophilized (below −50° C.) for 24 h using a freeze dryer (Alpha 1-2 LD plus, Martin christ, Germany). In addition, FTIR with attenuated total reflectance (ATR) was used to confirm the attachment of Triam molecules to AuNPs, and each spectrum was obtained using 32 scans with a resolution of 4/cm. In addition, the visual crystallinity and surface morphology of AuNPs and Triam-AuNP complexes were analyzed using TEM (F30, Tecnai, State missing, USA).

H₂O₂ Decomposition and O₂ Production Analysis

H₂O₂ (2.5 mM), AuNPs (10 μg/mL), Triam (5 μg/mL) and Triam-AuNPs were mixed in PBS at room temperature. Then, 50 μl of the solution was added to a solution of 100 μL of Ti (SO₄)₂ containing 1.33 mL of 24% Ti(SO₄)₂ and 8.33 mL of H₂SO₄ in 50 mL of deionized water after 24 hours. After the reaction was completed, the absorbance of the solution was measured at 405 nm to evaluate the H₂O₂ concentration. In addition, in order to observe O₂ bubbles in the Eppendorf tube, AuNPs, Triam, and Triam-AuNPs at the same concentration were mixed in 5 M H₂O₂ for 2 hours and monitored.

### Cell Culture and Cell Viability

FLSs were isolated by enzymatic dispersion of synovial tissues of arthritis patients (E. J. Nam et al., Arthritis Rheum., 65 (7): 1753-1763. 2013). Specifically, synovial tissue samples were obtained from RA and OA patients during joint surgery. The age of the arthritis patient was between 32 and 59 years old, and the obtained tissues were monolayer cultured. Informed consent was obtained from all patients, and the Ethics Committee of Eulji University approved the study of the present invention. FLSs were cultured in DMEM supplemented with non-heat-inactivated 10% FBS and 1% antibiotics under conditions of 5% CO₂ and 37° C., and FLSs were used for experiments at passages 3-7.

Preparation of Triam-AuNPs Labeled with Alexa Fluor 488

Triam-AuNPs labeled with Alexa Fluor 488 were prepared by mixing Triamcinolone and Alexa Fluor 488 (S11223, Molecular Probes, OR, USA, USA) with a pegylated AuNP solution in DW. The mixture was incubated overnight without light and the conjugation reaction was stopped by centrifugation to remove free Triamcinolone and Alexa Fluor 488. Triam-AuNPs labeled with Alexa Fluor 488 were washed three times with DW, and fluorescence was confirmed using a Victor 1420 Multilabel Counter (PerkinElmer, Waltham, MA, USA) at an excitation wavelength of 485 nm and an emission wavelength of 535 nm.

### Uptake Analysis of Triam-AuNPs in FLSs and J774 Cells

FLSs and J774 cells were seeded on poly-d-lysine-coated coverslips (5×10 4 cells) and incubated overnight. The cells were incubated various inhibitors at various concentrations (50 UM EIPA, 20 μM CPZ or 200 UM of GEN, Sigma) for 30 min at 37° C. to inactivate micropinocytosis, caveolae-mediated and clathrin-mediated pathway. Then, 0.5 μg/mL streptavidin- and Alexa 488-conjugated Triam-AuNPs (Alexa 488-Triam-AuNPs) were added to the cells and incubated at 37° C. for 6 hours. All samples were then fixed overnight in 4% paraformaldehyde in medium at 4° C. and signals from the various treatment groups were obtained using an LSM700 confocal microscope (Carl Zeiss) with the same settings.

### ROS Detection In Vitro

For in vitro ROS detection, cells were pretreated with experimental drugs (ie, PBS, AuNPs, Triam and Triam-AuNPs) at a concentration of 500 ng/mL for 24 h. Then, dihydroethidium bromide (DHE), a ROS probe, was added to the cells at a concentration of 400 nM, and incubated at 37° C. for 10 minutes. In the presence of ROS, the DHE is rapidly oxidized to form a bright product. ROS signals in the various treatment groups were obtained with the same settings using an LSM700 confocal microscope (Carl Zeiss). Red fluorescence intensity was quantified using ImageJ software.

### Real-Time PCR

To measure cytokine expression, RT-PCR (CFX 384, Bio-Rad, Hercules, CA, USA) was performed according to the manufacturer's protocol. Specifically, FLSs were pretreated with Triam and Triam-AuNPs for 1 hour and then stimulated with TNF-α (20 ng/ml) for 12 hours. Then, total RNA was isolated from cells (5×10⁵ cells/well in a total of 6-well plates) using TRIzol (15596018, Thermo Fisher Scientific) and first strand complementary DNA (cDNA) using RT premix (Promega) was synthesized. Reverse transcription conditions were carried out at 45° C. for 60 minutes and at 95° C. for 5 minutes. Briefly, in each final reaction tube, 2 μL of cDNA (1 μg), 1 μL of sense and antisense primer solutions (0.4 μM), 12.5 μL of SYBR Premix Ex Taq (Takara Bio Inc.) and 9.5 μL of dH₂O. The cycle threshold (Ct) value was calculated using the CFX96 Real-Time PCR Detection System (Bio-Rad) software, and the comparative Ct method (2ΔΔCt model) was used to calculate the relative fold change of gene expression. It was then normalized to mean GAPDH expression. The nucleic acid sequences of the primers used in the present invention are summarized in Tables 1 and 2 below.

TABLE 1
Information of nucleotide sequences
of primers

| | | SEQ |
|---|---|---|
| | | ID |
| Primer | Nucleotide Sequences (5′→3′) | Nos: |
| TNF-α F | CTC TTC TCC TTC CTG ATC GTG | 1 |
| TNF-α R | CCA GAG GGC TGA TTA GAG AGA | 2 |
| IL-1β F | GCT CGC CAG TGA AAT GAT G | 3 |
| IL-1β R | CAG AGG GCA GAG GTC CAG | 4 |
| IL-6 F | CCT CCA GAA CAG ATT TGA GAG TA | 5 |
| IL-6 R | TCA GGG GTG GTT ATT GCA TCT A | 6 |
| IL-4 F | ATG GGT CTC ACC TCC CAA C | 7 |
| IL-4 R | ATA TCG CAC TTG TGT CCG TG | 8 |
| IL-10 F | TGC CTT TAA TAA GCT CCA AGA G | 9 |
| IL-10 R | TCT TCA TTG TCA TGT AGG CTT C | 10 |
| Arg-1 F | AGG AAT TGG CAA GGT GAT GG | 11 |
| Arg-1 R | GTG AAA GAT GGG TCC AGT CC | 12 |
| iNOS F | TCT ACC AGG AGG AGA TGC TG | 13 |
| iNOS R | CCT GAA CAT AGA CCT TGG GC | 14 |
| MMP-1 F | GAG TTC CTG ACG TTG GTC AC | 15 |
| MMP-1 R | ACA GCA TCT TTT GGC AAA TC | 16 |
| MMP-3 F | TCC CAA GCA AAT AGC TGA AG | 17 |
| MMP-3 R | CAT TTG GGT CAA ACT CCA AC | 18 |
| GAPDH F | GTA TGA CAA CGA ATT TGG CTA CAG | 19 |
| GAPDH R | TCT CTC TCT TCC TCT TGT GCT CTT | 20 |

TABLE 2
Information of nucleotide sequences
of primers

| | | SEQ |
|---|---|---|
| | | ID |
| Primer | Nucleotide Sequences (5′→3′) | Nos: |
| TNF-α F | TGT CTA CTG AAC TTC GGG GT | 21 |
| TNF-α R | GAG GGT CTG GGC CAT AGA A | 22 |
| IL-1β F | CTG TTC TTT GAA GTT GAC GGA C | 23 |
| IL-1β R | GCG AGA TTT GAA GCT GGA TG | 24 |
| IL-6 F | CTT CAC AAG TCG GAG GCT TAA T | 25 |
| IL-6 R | AGT GCA TCA TCG TTG TTC ATA C | 26 |
| IL-4 F | ATC ATC GGC ATT TTG AAC GAG GTC | 27 |
| IL-4 R | ACC TTG GAA GCC CTA CAG ACG A | 28 |
| IL-10 F | GTC ATC GAT TTC TCC CCT GTG | 29 |
| IL-10 R | GTA GAC ACC TTG GTC TTG GAG | 30 |
| Arg-1 F | GCA GAG GTC CAG AAG AAT GG | 31 |
| Arg-1 R | ACA CAT AGG TCA GGG TGG AC | 32 |
| iNOS F | GGG AGC CAC AGC AAT ATA GG | 33 |
| iNOS R | TCA GCC TCA TGG TAA ACA CG | 34 |
| GAPDH F | TGC TGA GTA TGT CGT GGA GT | 35 |
| GAPDH R | AGA TGA TGA CCC TTT TGG CTC | 36 |

### Collagen-Induced Arthritis (CIA) Model

DBA/1 mice (male, 4-6 weeks old, body weight 20-25 g) were purchased from Orient Bio (Seoul, Korea) and the mice were subjected to a 12-hour light/dark cycle (lit at 6:30 AM) for 7-14 days. They were placed in a specific pathogen-free environment with temperature and humidity controlled and acclimatized before the experiment. All animal experiments were performed according to the Gachon University Laboratory Animal Care and Use Guide. Arthritis was also induced by tail-based intradermal injection of 2 mg/mL CFA (Chondrex). Animals with AIA-induced arthritis were randomly assigned to 6 groups (n=6) after the first signs of inflammation were observed on day 27.

### Atopic Dermatitis Model

BALB/c mice (female, 6-10 weeks old, body weight 16-22 g, n=5) were purchased from the Korea Laboratory Animal Center (Daejeon, Korea) and the mice were subjected to a 12-hour light/dark cycle for 7 days (6 am at 6:30 am). 30), and were accommodated in a specific pathogen-free environment with temperature and humidity controlled and acclimatized before the experiment. All animal experiments were performed according to the guide for the care and use of laboratory animals at Kyungpook National University. In addition, dinitrochlorobenzene (2,4-DNCB) and house dust mite extract (DFE) were alternately applied to induce atopic dermatitis. To put it simply, 30 μl of dinitrochlorobenzene is applied to the back twice in the first week after hair removal of the mouse, etc. From the second week to the third week, 30 μl of dinitrochlorobenzene is applied once and 30 μl of house dust mite (1 mg/mL) is applied twice. After the second week, Dex-AuNPs are applied to the induced area according to the concentration. Mice are sacrificed by completing the experiment on day 21.

### Psoriasis Model

BALB/c mice (female, 9-10 weeks old, weight 18-20 g, n=5) were purchased from the Korea Laboratory Animal Center (Daejeon, Korea), and the mice were subjected to a 12-hour light/dark cycle (6 am to 6 am) for 7 days and were acclimatized in a specific pathogen-free environment with temperature and humidity controlled. Aldara cream (5% imiquimod) was used to induce psoriatic dermatitis. Briefly describing the induction of psoriasis, the hair on the back of the mouse is removed using an epilator and a depilatory agent. And leave it for 2 days to get rid of the effect of the depilatory cream. And after that, Aldara cream (62.5 mg) is applied to the back of the mouse every day and 2 hours later, Dex-AuNPs are treated. Repeat this process for 7 days. The next day the mice are sacrificed.

Scoring of Arthritis and Treatment of RA Mice with Triam-AuNPs

The status of RA was assessed visually by grading paw swelling according to the following criteria: 0, no swelling; 1, inflammation and swelling in one toe; 2, more than one toe, but inflammation and swelling throughout the foot; 3, moderate swelling of the entire foot; 4, Severe erythema and swelling of the entire foot and ankle (H. E. Scales, et al., Rheumatology (Oxford) 55 (3): 564-572, 2016). A total score of four paws was recorded for each mouse as the arthritis index. After the RA model was established, mice scoring 3 points were randomly classified into the following 7 groups: saline, AuNPs, low-dose Triam (2 mg/kg), high-dose Triam (5 mg/kg), and low-dose Triam-AuNPs (2 mg/kg Triam) and high-dose Triam-AuNPs (5 mg/kg Triam) (n=5). Each treatment group was administered twice weekly for 3 weeks, 2 mg/kg or 5 mg/kg condition, and the arthritis index of each group was recorded over time. After 3 weeks of treatment, mice were sacrificed, and limbs were collected and histological analysis was performed.

### Tissue-Fluorescence Analysis

Histopathology was evaluated to determine the characteristics of RA including macrophage phenotype, synovitis and cartilage destruction. Specifically, on day 48, the mice were sacrificed and the ankle joint was recovered. The knee joint was fixed in 4% paraformaldehyde at 4° C. for 3 days, transferred to a descaling solution (Sigma), and left on an orbital shaker. Then, the tissue was decalcified at room temperature for 14 days, and the non-calcified ankle joint was embedded in paraffin and then cut into 3 μm using a microtome. For immunofluorescence histochemistry, tissue sections were dewaxed and TNF-α (Abcam, ab1793), IL-1β (Abcam, ab9722), IL-6 (Abcam, ab7737), INF-γ (Novus Biotech, NBP1), CD86 (Invitrogen, 13-0862-82), and Dectin-1 (Abcam, ab140039) were stained with primary antibodies. The secondary antibodies used in the present invention are goat anti-rat Alexa Fluor 488 (Abcam) for INF-γ, goat anti-mouse Alexa Fluor 488 (Abcam) for TNF-α, goat anti-mouse Alexa Fluor 568 for CD86 (Abcam), goat anti-rabbit Alexa Fluor 488 (Abcam) for IL-1β and IL-6 and goat anti-rabbit Alexa Fluor 568 (Abcam) for Dectin-1. The tissue sections were then stained with DAPI mounting solution and observed using a confocal fluorescence microscope (LSM700, Carl Zeiss) and EVOS M7000, Invitrogen. Fluorescence intensity was also quantified using ImageJ, Zen Lite (Zeiss) and Celleste (Invitrogen).

### Ethics

Synovial cells (FLSs) used in the present invention were isolated from rheumatoid arthritis patients at Kyungpook National University and osteo-degenerative arthritis patients at Nowon Eulji Medical Center (IRB numbers: 2052-040903 and 2017-10-009-005), respectively.

### Statistical Analysis

The significance of the difference between the mean values obtained in the two sample groups was analyzed using Student's t-tests. Differences were considered significant if the p-value was less than or equal to 0.05. Analysis of variance (ANOVA) followed by the Newman-Keuls 3 multiple comparison test was used to analyze the significance of differences between different sample means. Asterisks (*, ** and ***) indicate the meaning of the p values (<0.05, <0.01 and <0.001, respectively).

### Example 1: Physicochemical Properties

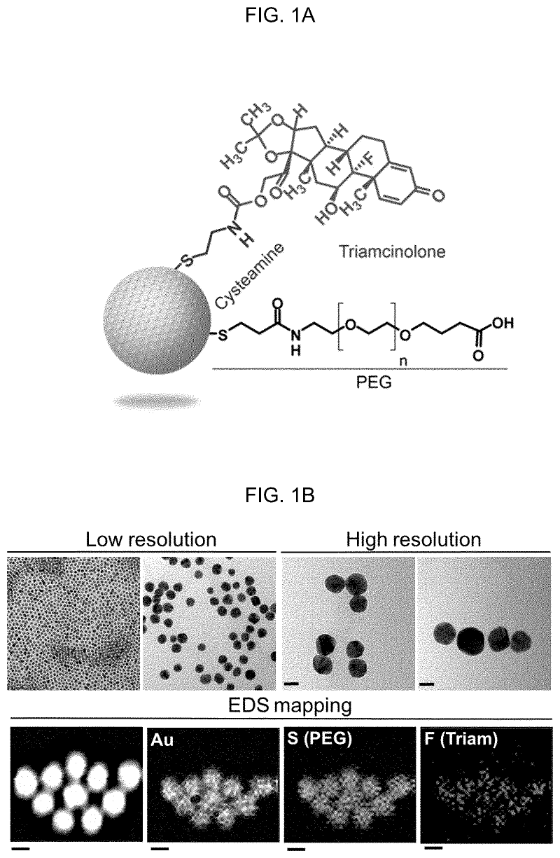

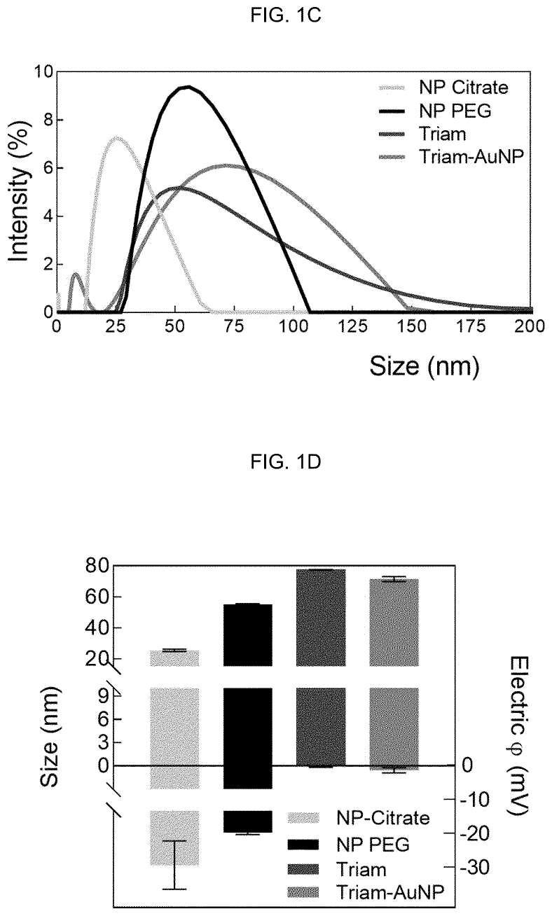

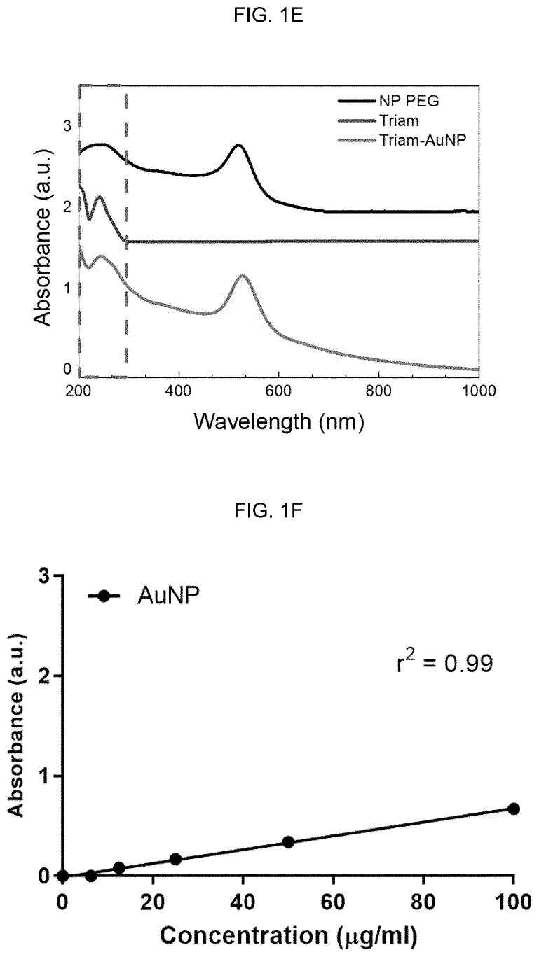

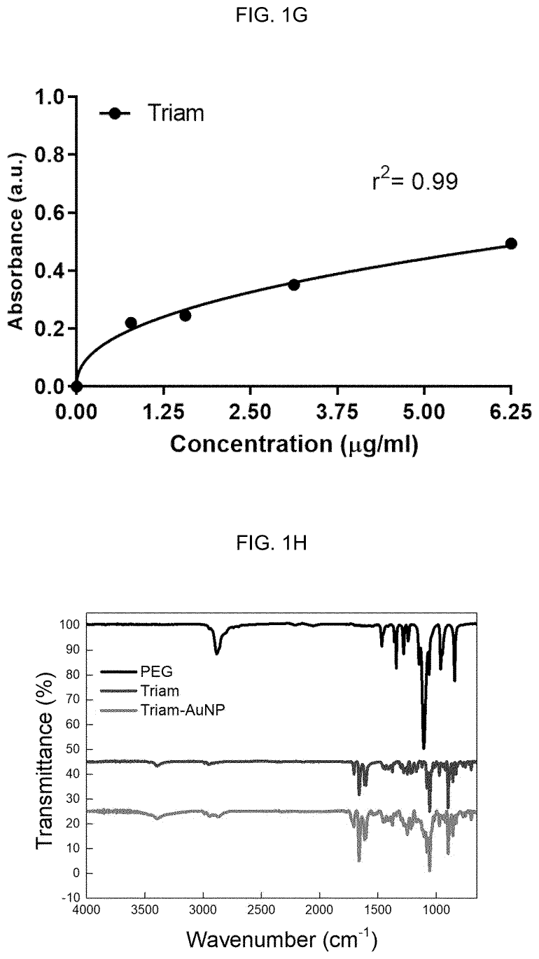

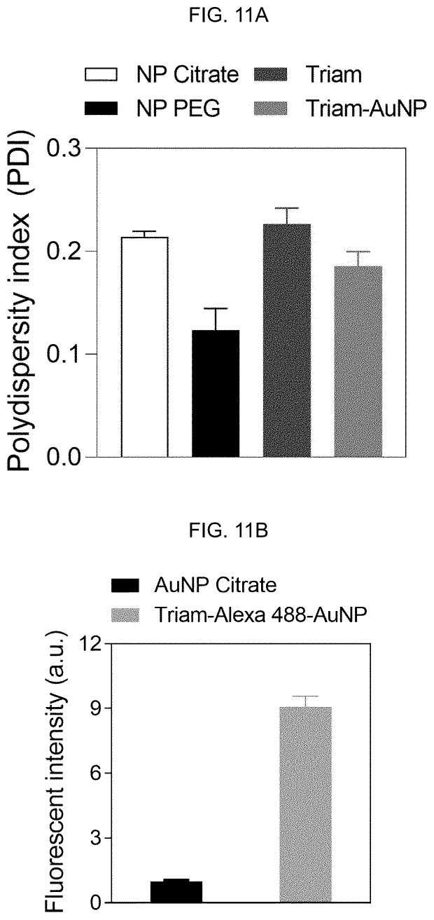

The present inventors covalently conjugated PEGylated AuNPs with Triamcinolone through cysteamine (FIG. 1A) and analyzed the chemical and physical properties of Triam-AuNPs. Specifically, a round image of the Triam-AuNP complex having a diameter of 20 nm was confirmed through a transmission electron microscope (TEM, FIG. 1B). In addition, through energy-dispersive X-ray spectroscopy (EDS), the signals of Au, S (sulfur, PEG) and F (fluorescence, Triam) at the same position were clearly indicated, and through the exact match of each chemical, Triamcinolone conjugated to pegylated AuNPs was observed. In addition, the hydrodynamic sizes of free AuNPs, free Triam, pegylated AuNPs and Triam-AuNPs were 25, 75, 55 and 71 nm, respectively (FIGS. 1C and 1D). In addition, the polydispersity index (PDI) of all samples tested through the polydispersity index (PDI) was less than 0.5, and after 10 days of synthesis, it was confirmed that Triam-AuNPs had greater solubility than other formulations (FIG. 11A). Analysis of the electric potential caused by the surface charge showed that both the free Triam and Triam-AuNPs approached neutral charges, whereas the pegylated AuNPs showed a negative charge (FIG. 1D). In addition, as a result of analyzing the absorbance through ultraviolet-visible (UV-vis) spectroscopy, AuNPs showed a peak at 520 nm and Triam at 230 nm (FIGS. 1E to 1G), and AuNP: Triam ratio was 34:1 as a mass ratio (see Table 3). In addition, the presence of the same oscillating infrared (IR) peak between Triam and Triam-AuNPs was confirmed through Fourier transform infrared (FT-IR) spectroscopy, confirming the conjugation of Triam and AuNPs (FIG. 1H).

TABLE 3
AuNP:Triam ratio

| | NP:Triam(20 mg:10 mg) |
|---|---|
| Mass of AuNP after conjugation with | 16.76 |
| triamcinolone 9) | |
| Normalized loss percentage of Au (%) | −16.20 |
| Mass of conjugated triamcinolone (μg) | 489 |
| Percentage of conjugated | 2.9 |
| triamcinolone per Au (%) | |
| Final AuNP:Triam ratio | 34:1 |

### Example 2: Nanodrug Uptake Pathway of Inflammatory FLSs and M1 Macrophages

When NPs are surrounded by cells, the two main factors influencing internalization are the physicochemical properties of the NPs and the cell type (S. Behzadi et al., Chem. Soc. Rev., 46 (14): 4218-4244, 2017). Endocytosis (eg, micropinocytosis, clathrin- and caveolae-mediated endocytosis) is a major internalization pathway during NP internalization (Y. K. Lee et al., J. Mater. Chem. B, 4 (9): 1660-1671, 2016). In order to confirm the specific intracellular mechanism responsible for the uptake of Triam-AuNPs, the present inventors treated cells with inhibitors specific inhibiting three types of endocytosis, 5-(N-ethyl-N-isopropyl) amiloride (EIPA) as a micropinocytosis inhibitor, chlorpromazine (CPZ) as a clathrin-mediated endocytosis inhibitoromazine) and genistein (GEN) as a caveolae mediated endocytosis inhibitor, respectively. In addition, streptavidin-Alexa 488-conjugated Triam-AuNPs (Triam-Alexa 488-AuNPs) were used to investigate the intracellular uptake of Triam-AuNPs in FLSs and macrophages.

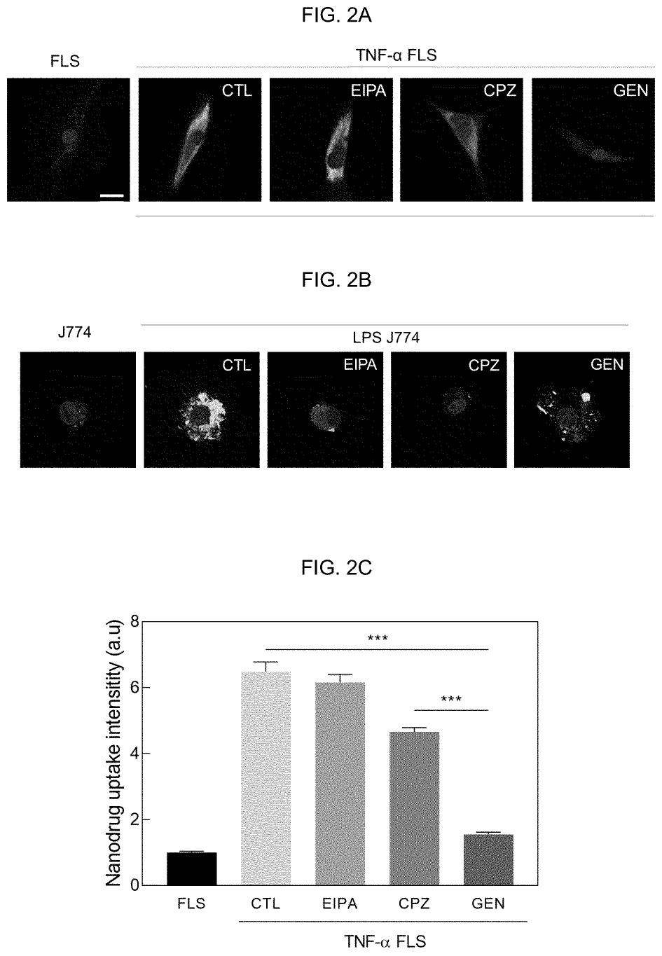

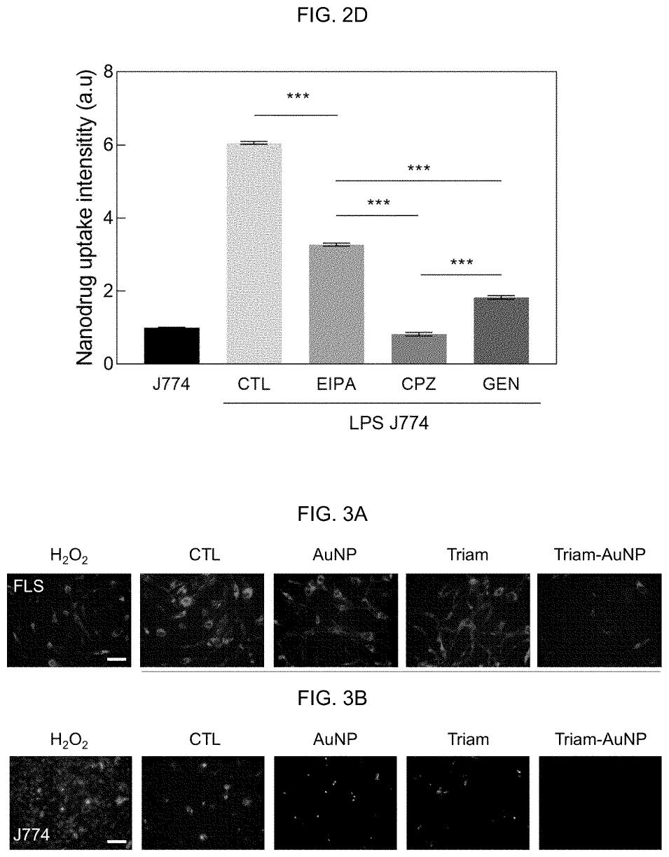

As a result, non-covalent Alexa 488 conjugation to Triam-AuNPs was confirmed with a fluorescence spectrophotometer, and strong absorbance was detected at 490 nm (FIG. 11B). As a result of analyzing the intracellular uptake of Triam-AuNPs after 6 hours of incubation, TNF-α-stimulated FLSs exhibited higher uptake than normal FLS (FIG. 2A). Specifically, the uptake pathway of Triam-AuNPs was highly dependent on caveole (70%)- and clathrin (30%)-mediated endocytosis in TNF-α-stimulated FLSs.

On the other hand, Triam-AuNP uptake was found to include all three uptake pathways in M1 macrophages, and among them, macropinocytosis and clathrin-mediated intracellular uptake pathways were confirmed to be significant (FIGS. 2B and 2D).

### Example 3: H₂O₂-Scavenging Effect of AuNPs and Intracellular Reactive Oxygen Species (ROS) Analysis

We performed H₂O₂-scavenging effect of AuNPs and intracellular ROS analysis before pro-inflammatory cytokine secretion assay. Specifically, after treatment with Triamcinolone and AuNPs under physiological conditions (pH 7.4), concentration of H₂O₂ was analyzed.

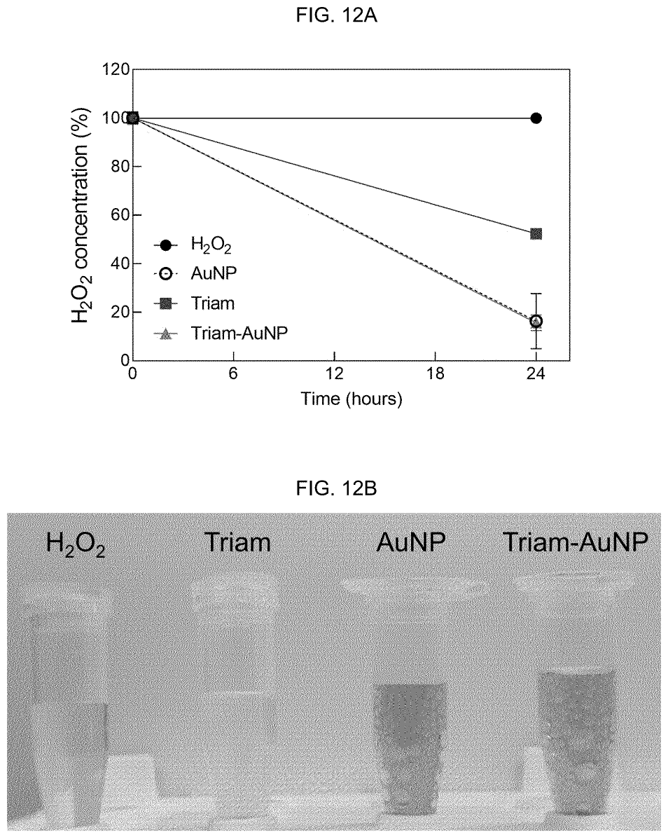

As a result, H₂O₂ concentration was reduced by about 48% after 24 hours of Triam treatment, but Triam-AuNPs and AuNPs (same AuNPs concentration) treatment showed that most of H₂O₂ was decomposed after 24 hours. These results suggest that AuNPs themselves exhibit a strong catalytic effect (FIG. 12A). In addition, after treatment with AuNPs and Triam-AuNPs in H₂O₂ solution and incubation for 2 hours, O₂ generating function was significantly increased at pH 7.4, and abundant O₂ bubbles were confirmed in both AuNP and Triam-AuNP groups (FIG. 12B).

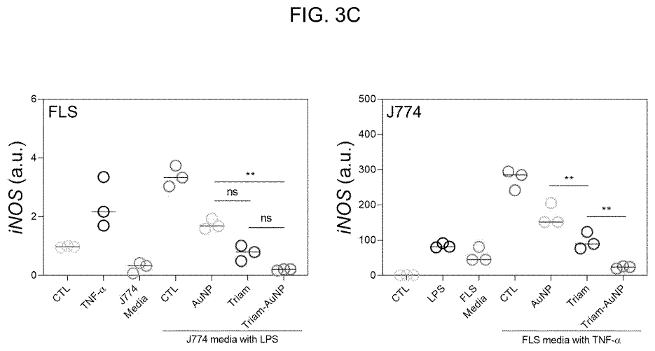

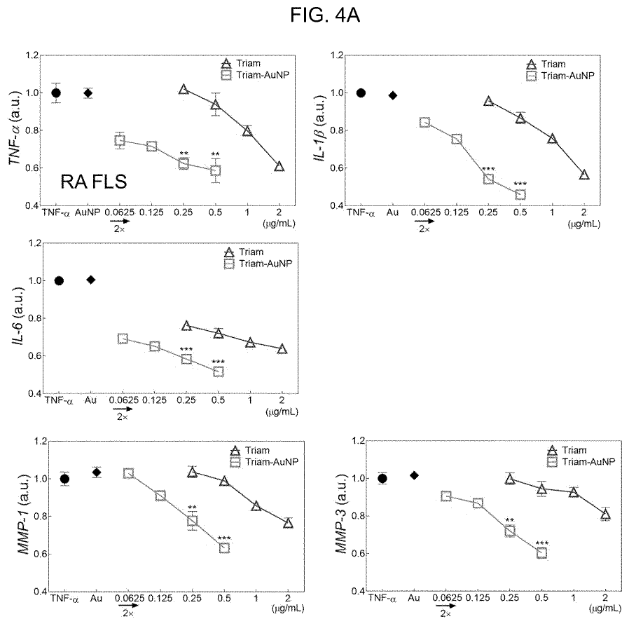

In addition, intracellular ROS generated by FLSs and J774 cells were analyzed after treatment with the drug for 24 hours. To this end, TNF-α (20 ng/mL) was added to activated FLSs, and lipopolysaccharide (LPS, 50 ng/mL) was added to macrophages (J774). As a result, it was found that the Triam-AuNPs of the present invention significantly inhibited ROS activity in both FLSs and M1 macrophages (FIGS. 3A and 3B). However, despite the fact that AuNPs largely ablated H₂O₂, the source of ROS activity, the intracellular ROS-scavenging effect in FLSs and macrophages was quite limited. Therefore, it was confirmed that the Triam-AuNPs conjugation of the present invention is very effective in reducing the ROS activity associated with the inflammatory response of FLSs and J774 cells. In addition, gene expression of iNOS, which was directly linked to the generation of highly reactive ROS, was significantly inhibited by Triam-AuNPs in activated FLSs and M1 macrophages (FIG. 3C).

### Example 4: Low-Dose Anti-Inflammatory Effects of FLSs (RA and OA Patients)

Considering the side effects induced by repeated steroid injections, a low-dose and effective steroid anti-inflammatory agent is an advantageous approach (J. P. Raynauld et al., Arthritis Rheum., 48 (2): 370-377, 2003). Therefore, in order to investigate the effect of the developed low-dose Triam-AuNPs compared to the effect of the relatively high-dose Triamcinolone, the present inventors analyzed expression level of TNF-α, IL-1β, IL-6 and MMP, which are major inflammatory markers for investigating the inhibition of inflammation in FLSs.

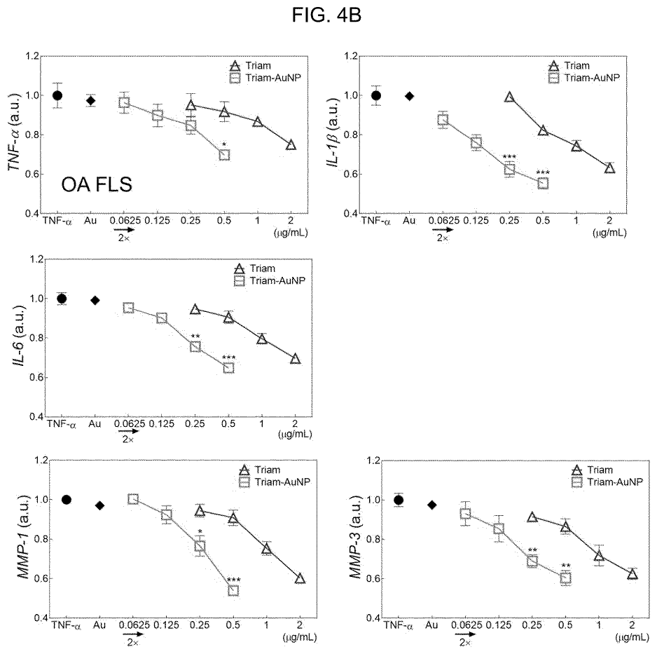

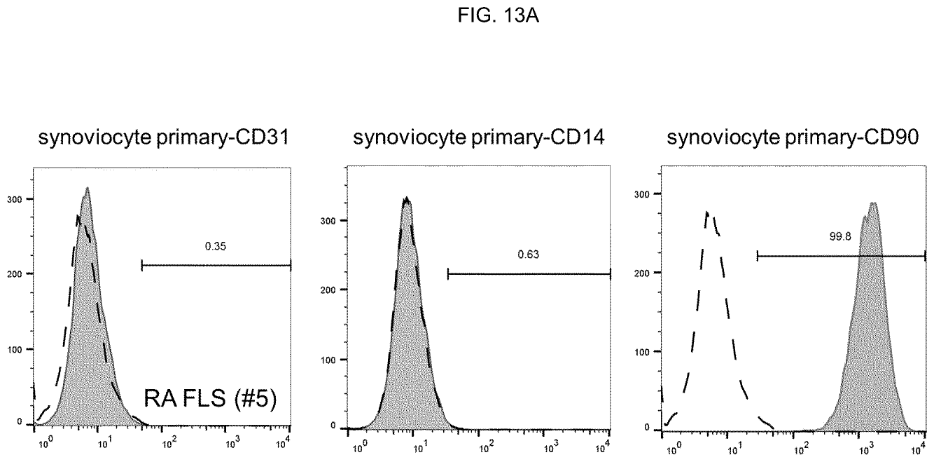

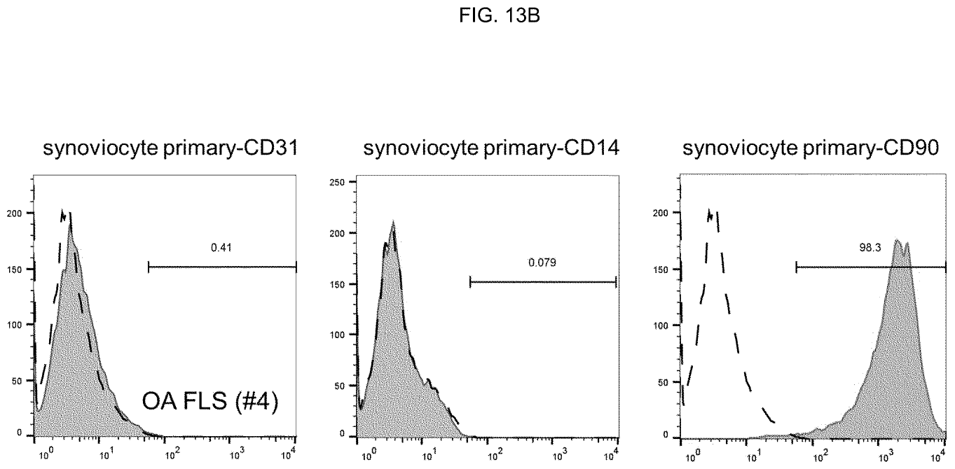

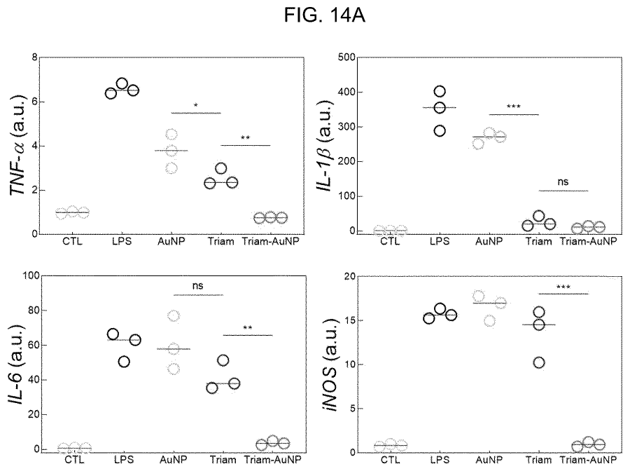

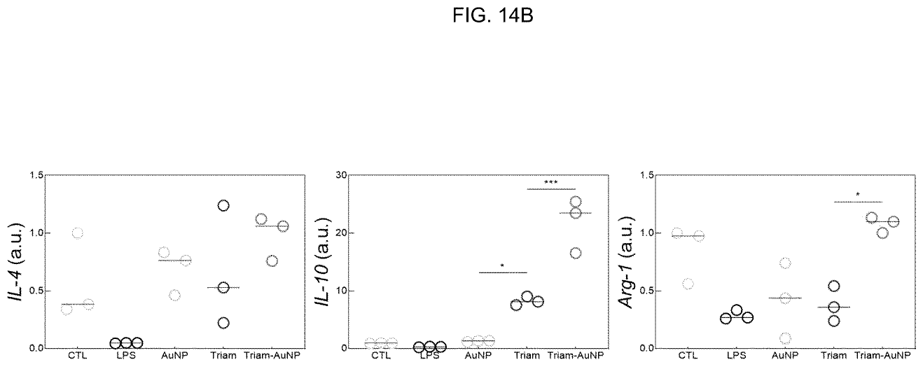

As a result, RA patient-derived primary FLSs and OA-generated cartilage tissue-derived FLSs (FIGS. 13A and 13B) exhibited the same pro-inflammatory characteristics (FIGS. 4A and 4B). Analysis of inflammatory cytokine (i.e., TNF-α, IL-1β, IL-6, MMP-1, and MMP-3) levels showed that Triam-AnNP at a concentration at least 4 time lower than triamcinolone alone showed higher therapeutic effect than triamcinolone alone treatment group in TNF-α-stimulated FLSs (from RA and OA patients) (FIGS. 14A and 14B).

### Example 5: Repolarization of FLSs by Nanodrugs

Recently, the role of macrophages in inflamed synovial membranes has been of great interest due to the inflammatory effects of these cells during pathogens of RA and OA in joints (A. Kennedy et al., Front. Immunol., 2:52, 2011). The present inventors therefore investigated the stimulation of FLS pro-inflammatory activity by activated M1 macrophage cytokines (from LPS-treated J774 cells to conditioned medium).

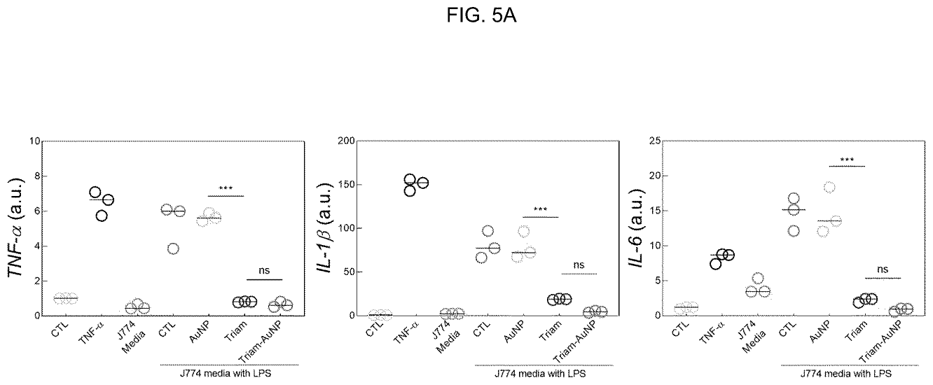

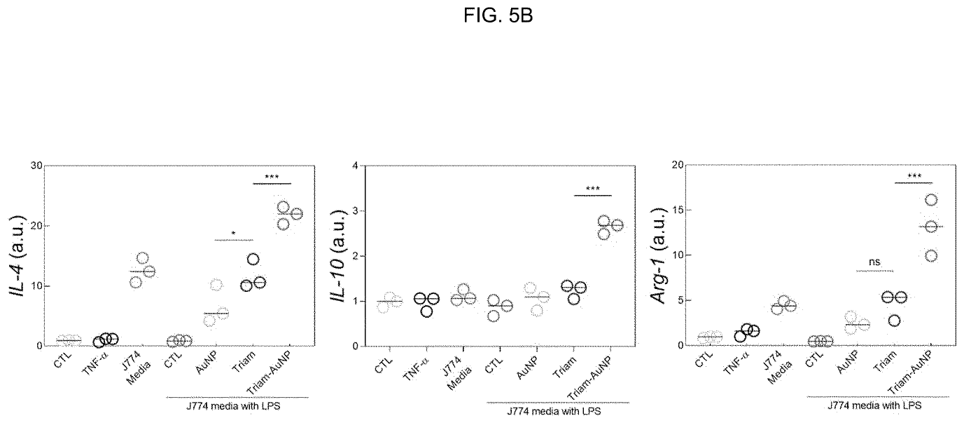

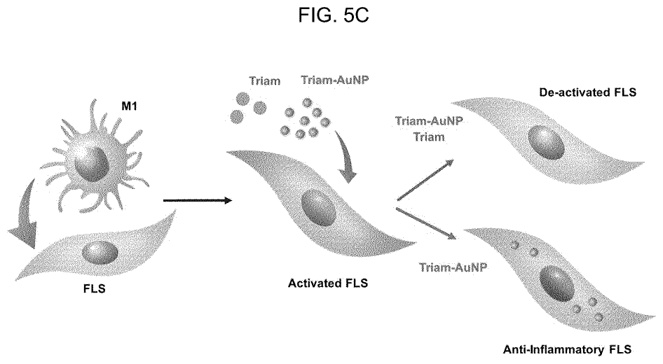

As a result, the levels of major pro-inflammatory cytokines (TNF-α, IL-1β and IL-6) in FLSs were significantly increased by treatment with M1 medium (FIG. 5A). These results suggest that M1 macrophages significantly affect the activation of FLSs in the inflammatory synovial membrane. In addition, treatment with Triamcinolone and Triam-AuNPs effectively reduced the level of FLSs pro-inflammatory cytokines (FIG. 5A). However, compared to Triamcinolone, Triam-AuNPs induced significantly higher increases in anti-inflammatory mRNA (IL-4, IL-10 and Arg-1) synthesis by M1-activated FLS (FIG. 5B). These results suggest that FLSs inflammatory repolarization (expressed as IL-4, IL-10 and Arg-1 expression) occurred only in the Triam-AuNPs group but not in the Triam-only group (FIG. 5C).

### Example 6: Macrophage Repolarization by Nanodrugs

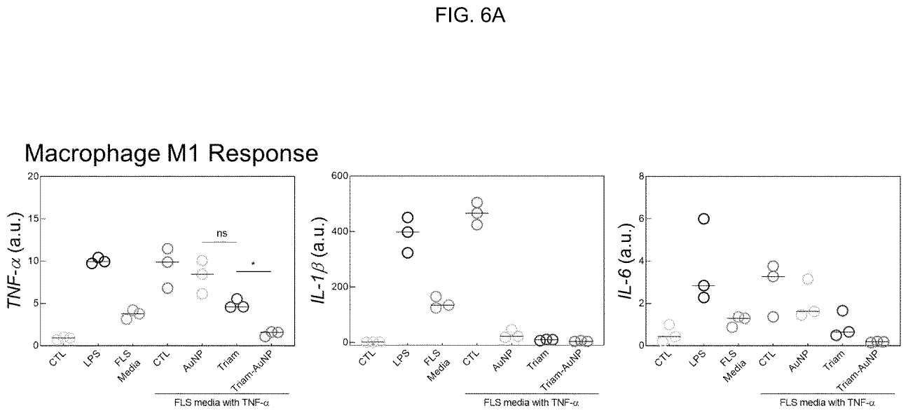

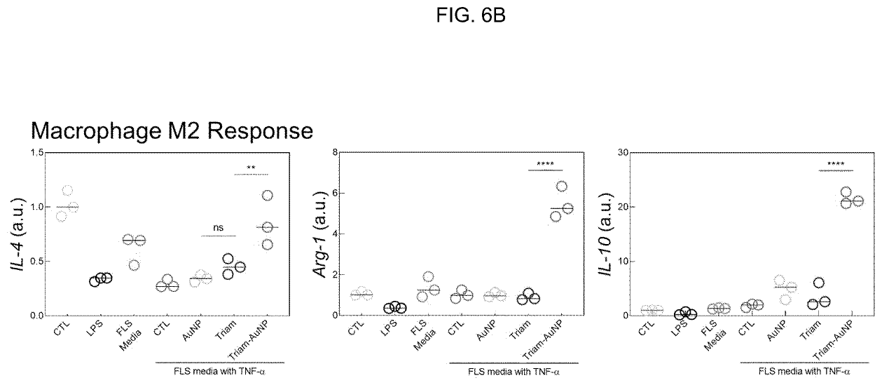

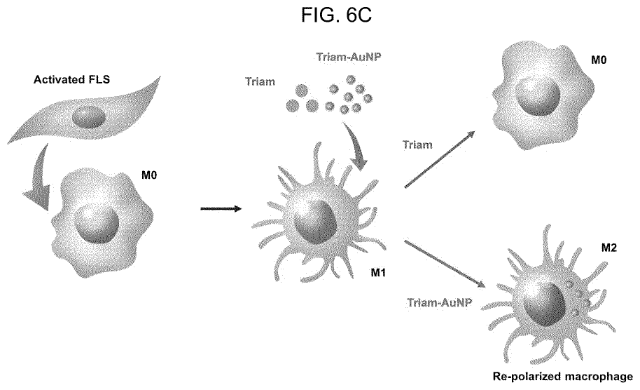

Macrophages of the synovium can stimulate pro-inflammatory FLSs activity by infiltrating inflamed tissues and increasing pro-inflammatory cytokine levels (M. Asif Amin et al., Semin. Immunopathol., 39 (4): 385-393, 2017). For this reason, modulating macrophage differentiation (ie, repolarization) is important in alleviating arthritis disease (J. Kim et al., ACS Nano, 13 (3): 3206-3217, 2019). Therefore, the present inventors investigated macrophage repolarization (from M1 to M2 phenotype) in order to determine way of converting activated M1 macrophages into M2 macrophages with anti-inflammatory properties after treatment with Triam-AuNPs, and how macrophages are activated by FLSs (by the addition of activated FLSs medium to macrophage medium). M1 polarization occurred after treatment with TNF-α-activated FLSs medium (indicated by TNF-α, IL-1β and IL-6 expression), but inflammatory marker levels were decreased significantly by Triam and Triam-AuNPs treatment (FIG. 6A). However, the M2 response (IL-4, Arg-1 and IL-10 expression) was significantly increased only by Triam-AuNPs treatment, and Triam alone treatment did not induce noticeable M2 marker expression (FIGS. 6B and 6C). Also, in the absence of FLS medium, macrophages activated by LPS (M1) were repolarized into M2 macrophages (expressing IL-4, IL-10 and Arg-1) by treatment with Triam-AuNPs (FIGS. 14A and 14B).

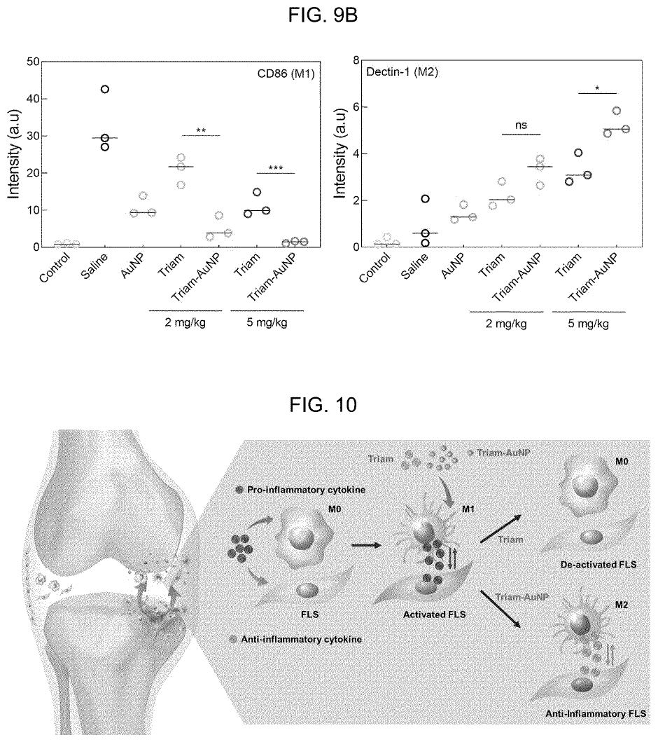

Based on the above results, the present inventors confirmed that macrophage repolarization (from M1 to M2 phenotype) was induced only by Triam-AuNPs (FIG. 6C). It is also thought that macrophage repolarization (via the interaction between FLS and macrophages) could significantly downregulate the subsequent activation of FLSs in the inflamed synovial membrane (FIG. 10).

### Example 7: Therapeutic Effect of Nanodrugs in CIA-Induced Mice

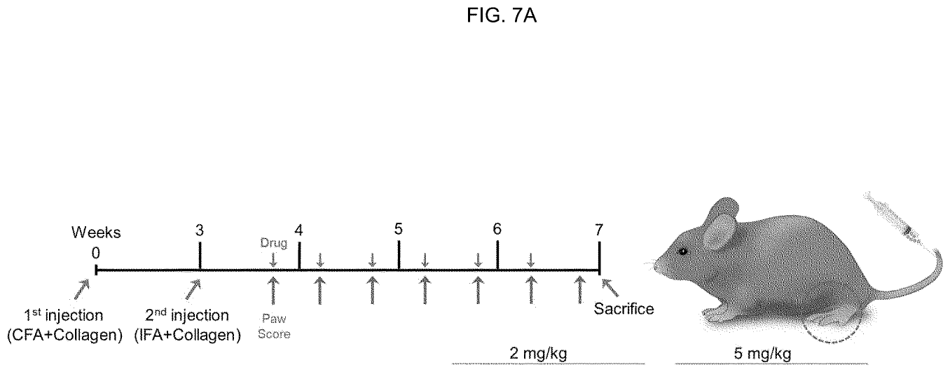

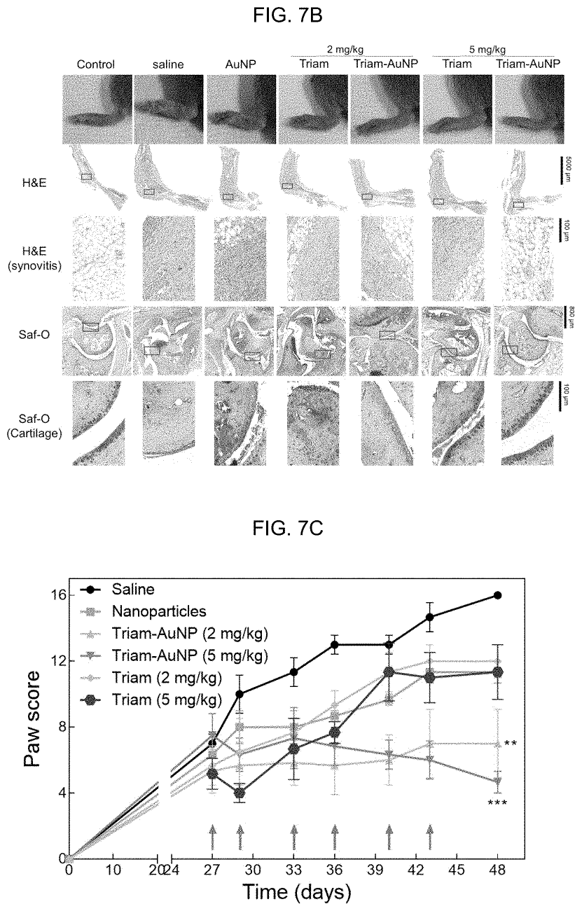

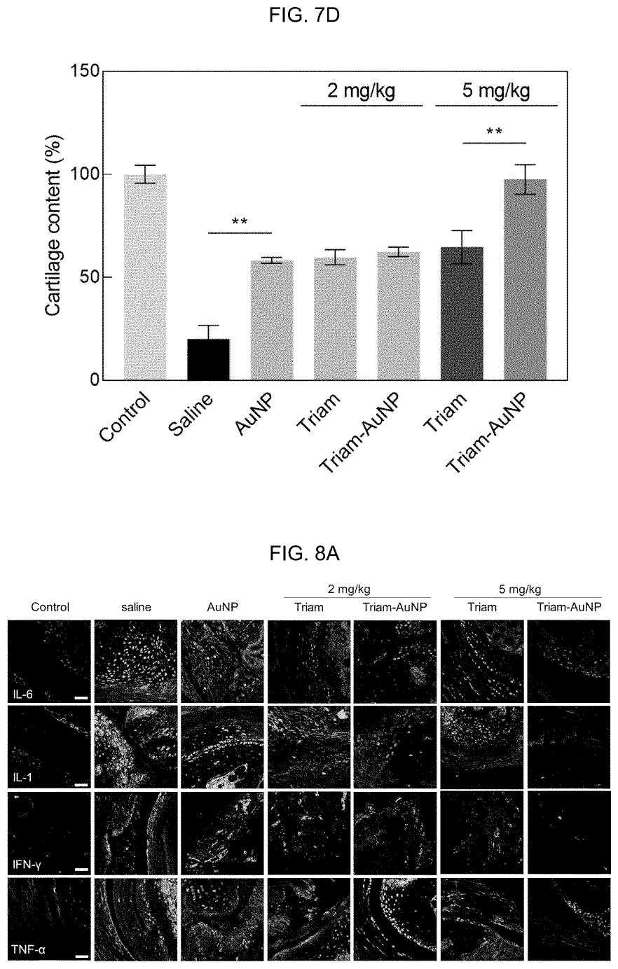

The present inventors established CIA-induced mice through intradermal injection of complete Freund's adjuvant (CFA) to evaluate the in vivo effect of Triam-AuNPs on RA treatment (FIG. 7A) (L. Bevaart et al., Arthritis Rheum., 62 (8): 2192-2205, 2010). The therapeutic efficacy of Triam-AuNPs was confirmed by paw swelling evaluation, histological image analysis and cartilage regeneration analysis.

As a result, the paw edema and paw score of the group treated with phosphate-buffered saline (PBS) significantly increased after 48 days, whereas the group treated with low-dose (2 mg/kg) and high-dose (5 mg/kg) Triam-AuNP showed significantly decreased symptoms (FIGS. 7B and 7C). However, Triam (2 mg/kg or 5 mg/kg) and AuNPs (at the same dose as Triam-AuNP-treated group) alone did not show sufficient effect in mice.

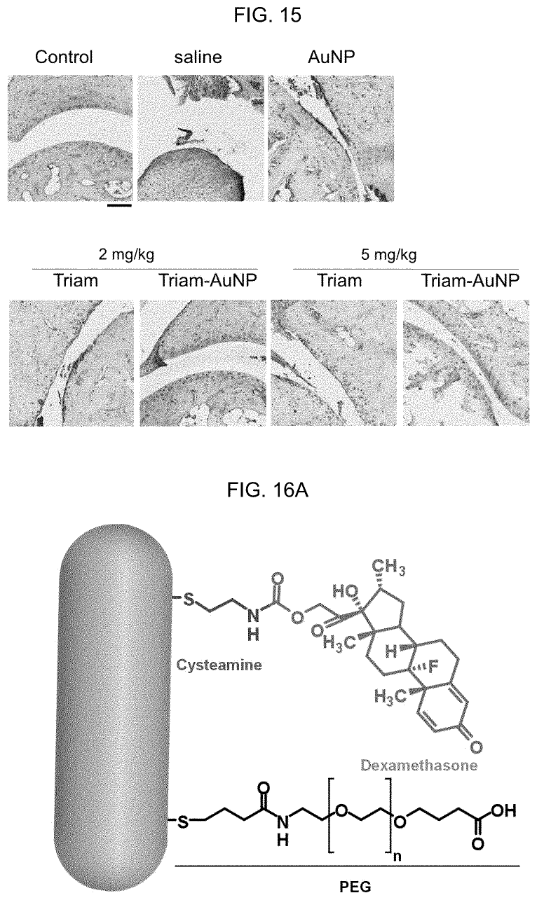

In addition, the present inventors evaluated the anti-inflammatory therapeutic effect of Triam-AuNPs according to an embodiment of the present invention by histological and immunohistochemical analysis. As a result, synovial inflammation was confirmed in the PBS-treated group in which the mouse ankle joint was stained with hematoxylin and eosin (H&E), but the Triam-AuNP combination treatment group showed significantly reduced synovial inflammation than the Triam alone treatment group (2 mg/kg and 5 mg/kg) (FIG. 7B). In addition, as a result of observing the ankle joint through safranin-O staining, greater cartilage destruction was confirmed in the PBS-treated group than in the control group. Cartilage showed better recovery in mice treated with Triam-AuNPs compared to mice treated with AuNPs or Triam alone (FIGS. 7B and 7D). In particular, expression of CD248 in injured ankle joints is evidence of upregulation of IL-1β and TNF-α (V. Pascual et al., J. Exp. Med., 201 (9): 1479-1486, 2005). Increased expression of CD248 represents an aggressive phenotype that invades the extracellular matrix and exacerbates joint damage in RA. Accordingly, the present inventors observed that there were almost no positively stained cells in the synovial membrane of the Triam-AuNPs treated group compared to the PBS- and Triam-treated groups through immunohistochemical analysis of CD248 (FIG. 15).

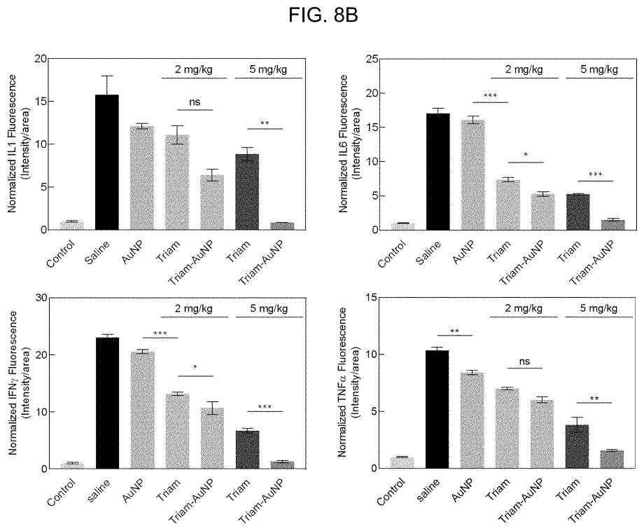

In addition, as a result of observing the fluorescence intensity to evaluate the expression level of inflammatory cytokines in the ankle joint, the expression of major inflammatory cytokines (IL-6, IL-1, INF-γ and TNF-α) in the Triam-AuNPs treatment group was found to be decreased (FIGS. 8A and 8B). The above results suggest that the treatment of Triam-AuNPs of the present invention effectively relieves inflammation and promotes cartilage regeneration in the inflammatory synovial membrane by increasing the anti-inflammatory response of FLSs and M2 repolarization of macrophages.

### Example 8: Induction of Macrophage Repolarization in the Synovial Membrane by Nanodrugs

The present inventors analyzed the proportion of M1 and M2 macrophages using immunofluorescence to investigate the repolarization of synovial macrophages in the ankle joint (J. Kim et al., ACS Nano 13 (3): 3206-3217, 2019).

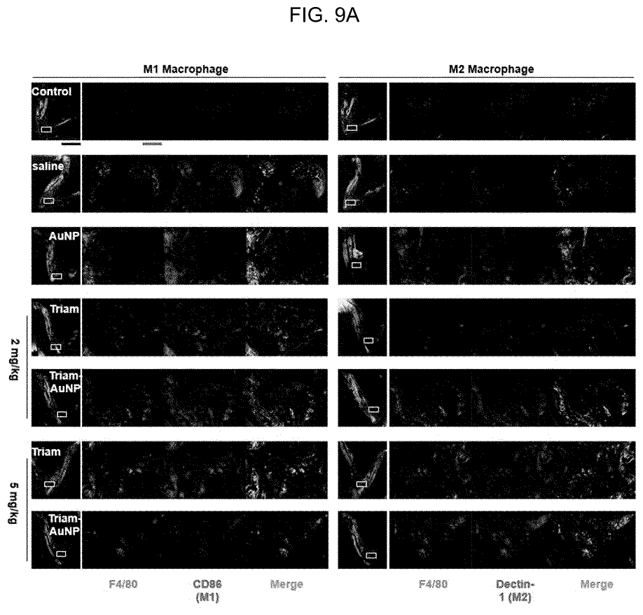

As a result, treatment with Triam-AuNPs regulated macrophage repolarization from M1 to M2 phenotype (FIGS. 6B and 9). In an in vivo experiment (in the CIA model), the same trend of macrophage repolarization was confirmed using fluorescence histochemical images (FIG. 9A). The above results clearly suggest that the treatment with Triam-AuNPs (5 mg/kg) of the present invention reduced the level of CD86 (M1 marker) and reversely increased the level of Dectin-1 (M2 marker) (FIGS. 9A and 9B). However, Triam treatment (2 mg/kg and 5 mg/kg) reduced the level of the M1 marker, but did not sufficiently restore the level of the M2 marker (FIGS. 6B and 6C). Therefore, synovial macrophages were differentiated into M2 macrophages by effectively regulating macrophage repolarization in the inflammatory synovial membrane when treated with Triam-AuNPs according to an embodiment of the present invention (FIGS. 9A and 9B).

### Example 9: Chemical and Physical Properties of Dex-AuNPs

The present inventors covalently conjugated PEGylated AuNPs with Dex through cysteamine (FIG. 16A) and analyzed the chemical and physical properties of Dex-AuNPs. Specifically, a columnar image of the Dex-AuNPs complex having a diameter of 10×40 nm (1:4 ratio) was confirmed through a transmission electron microscope (TEM) (FIG. 16B). In addition, the signals of Au, S (PEG & Cysteamine) and F (Dexamethasone) at the same position were clearly shown through energy-dispersive X-ray spectroscopy (EDS), and Dex was pegylated through the exact match of each chemical. It was observed that they were conjugated to AuNPs. In addition, the hydrodynamic sizes of pegylated AuNPs, free Dex and Dex-AuNPs were 28, 125 and 38 nm, respectively (FIGS. 16C and 16D). In addition, the polydispersity index (PDI) of all samples tested through the polydispersity paper (PDI) was less than 0.5, and the analysis of the electric potential caused by the surface charge showed that both free Dex and Dex-AuNPs were neutrally charged, whereas the pegylated AuNPs exhibited a negative charge (FIG. 1D). In addition, as a result of analyzing the absorbance through ultraviolet-visible (UV-vis) spectroscopy, AuNPs exhibited a peak at 800 nm and Dex at 242 nm (FIG. 16E).

### Example 10: Therapeutic Effect of Dex-AuNPs in Atopic Dermatitis Mice

The present inventors established atopic dermatitis mouse model by induced through repeated exposure to dinitrochlorobenzene and house dust mite extract to evaluate the in vivo effect of Dex-AuNPs on the treatment of atopic dermatitis. For the therapeutic efficacy of Dex-AuNPs, macroscopic observation, skin thickness measurement, epidermal and dermal thickness through histopathology, serum immunological response, and cytokine expression levels in tissues were measured.

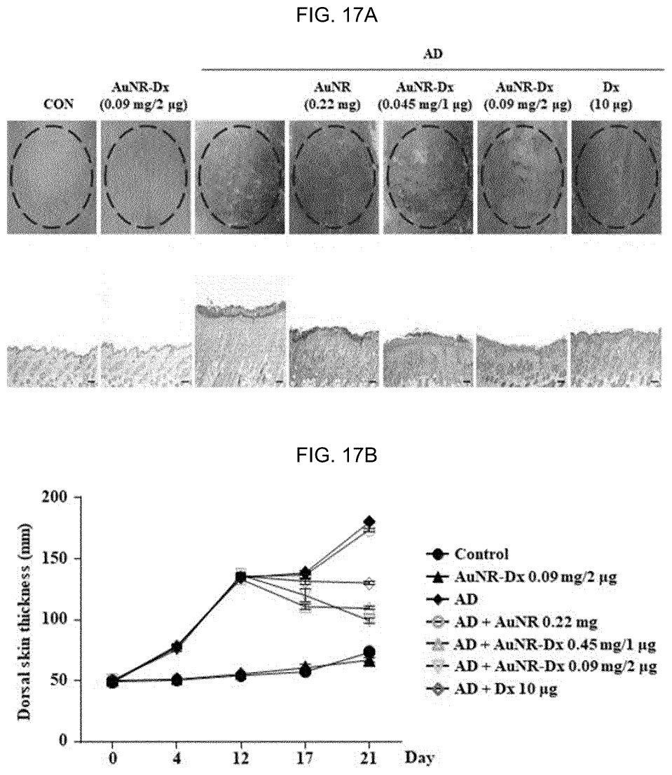

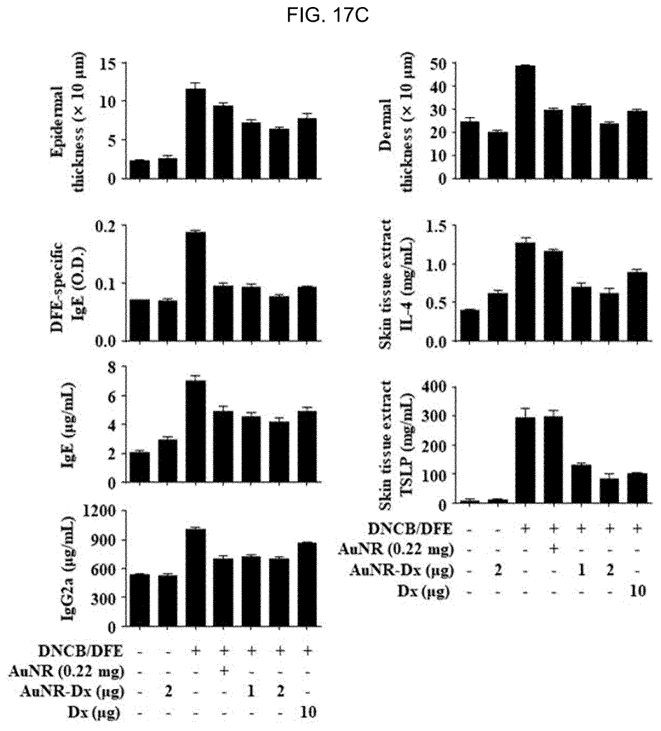

As a result, superficial phenomena of keratosis and erythema and histopathological analysis showed epidermis and dermis thickening and keratosis in atopic dermatitis-induced mice repeatedly exposed to dinitrochlorobenzene and house dust mite extract. In addition, the ELISA results of serum immunoglobulins IgE, IgG2a, and DFE-speicif IgE were also elevated, and the cytokines IL-4 and TSLP in tissues were also increased. On the other hand, mice to which Dex-AuNPs were applied showed erythema and keratosis and decreased skin thickness. In addition, as a result of histopathological analysis, epidermal and dermal thickness and keratosis were decreased, and the ELISA results of serum immunoglobulins IgE, IgG2a, and DFE-specific IgE were also decreased. In particular, the cytokines IL-4 and TSLP in tissues were also significantly reduced (FIGS. 17A, 17B and 17C). The above results suggest that the treatment of Dex-AuNPs of the present invention effectively reduces symptom relief of atopic dermatitis through immunological response.

### Example 11: Psoriasis Treatment Efficacy by Dex-AuNPs

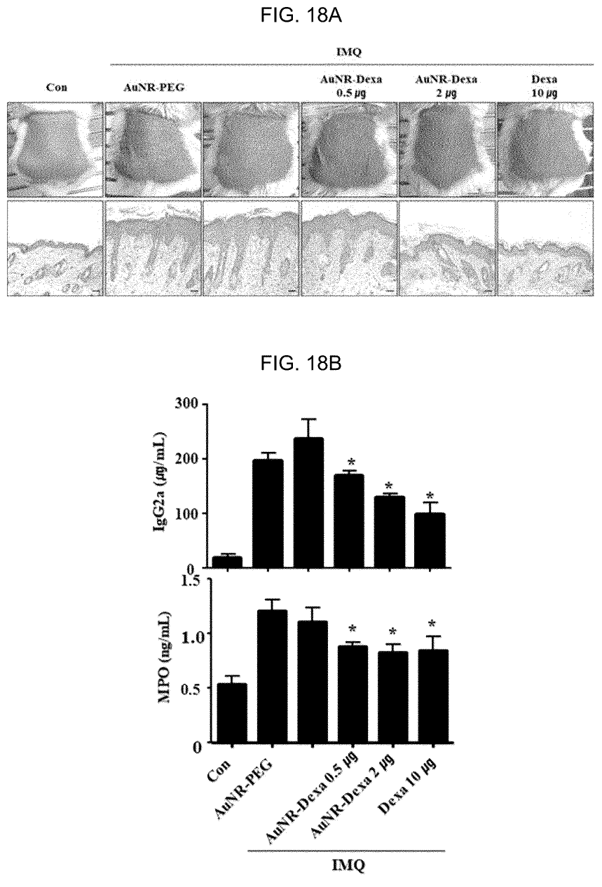

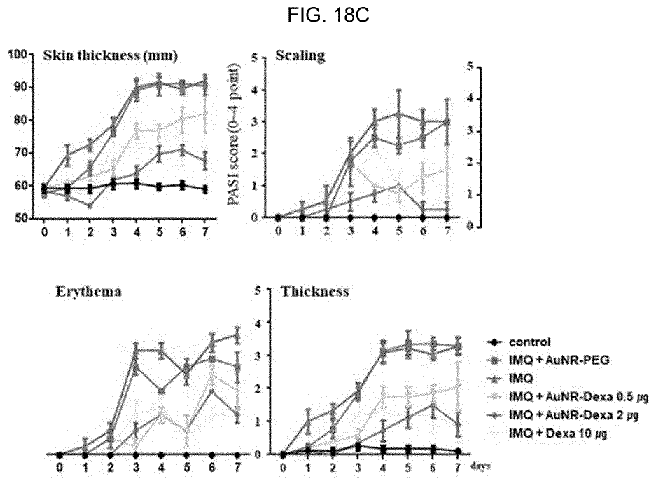

The present inventors established a psoriatic dermatitis model by continuously treating Aldara cream (62.5 mg) to confirm the in vivo effect of Dex-AuNPs on the treatment of psoriatic dermatitis. To confirm the therapeutic effect of Dex-AuNPs in psoriatic dermatitis, macroscopic observation, histopathological confirmation and immunological response, and psoriasis regional severity index were checked. As a result, it was confirmed that, when Aldara Cream was repeatedly treated, keratin, erythema, and skin thickness increased in the skin of mice with psoriatic dermatitis, and the serum immunoglobulin IgG2a, myeloperoxidase (MPO), and psoriasis regional severity index increased. However, in mice treated with Dex-AuNPs, keratin, erythema, skin thickness, immunoglobulin, and psoriasis regional severity index were all decreased (FIGS. 18A, 18B and 18C). The above results suggest that the Dex-AuNPs of the present invention can alleviate and treat symptoms of psoriatic dermatitis.

In conclusion, the triamcinolone-gold nanoparticle (Triam-AuNPs) complex, which is a novel inflammatory disease treatment agent of the present invention, reduces pro-inflammatory responses and anti-inflammatory responses of synovial cells (FLSs) through repolarization of macrophages from M1 to M2 phenotype, effectively regulating the expression of pro-inflammatory and anti-inflammatory cytokines in FLSs, and effectively repolarizing macrophage activity in the inflammatory synovial membrane. In addition, since it is effective in skin diseases such as psoriasis and atopic dermatitis, it can be used as a new drug candidate for treating inflammatory diseases.

The research involved in the present patent application is supported by the National Research Foundation in Korea with grant numbers, 2020M3A9D3038894 and 2019R1A2C1085986.

Although the present invention has been described with reference to the above-described embodiment, it will be understood that this is merely exemplary, and that those skilled in the art can make various modifications and equivalent other embodiments therefrom. Therefore, the true technical protection scope of the present invention should be determined by the technical spirit of the appended claims.

What is claimed is:

1. A method of treating inflammatory disease in a subject comprising administrating therapeutically effective amount of a gold nanoparticle-steroid complex in which a steroid anti-inflammatory agent is loaded on the surface of gold nanoparticle (AuNPs) coated with PEG having a number average molecular weight of 3,000 Da to 5,000 Da by covalent or non-covalent bond to the subject.

2. The method of claim 1, wherein the steroid anti-inflammatory agent is covalently bound to the gold nanoparticles coated with PEG by a thiol functionalized compound.

3. The method of claim 1, wherein the thiol-functionalized compound is cystamine, thiol-PEG, glutathione, cysteine or mercaptopropionic acid.

4. The method of claim 1, wherein the steroid anti-inflammatory agent is triamcinolone, hydrocortisone, prednisolone, betamethasone, or dexamethasone.

5. The method of claim 1, wherein the inflammatory disease is arthritis, psoriasis, or atopic dermatitis.

6. The method of claim 5, wherein the arthritis is osteoarthritis or rheumatoid arthritis.

7. The method of claim 1, wherein the loading ratio of the steroid anti-inflammatory agent to the PEG-coated gold nanoparticles is 6:4 to 7:3.

8. The method of claim 1, wherein the PEG-coated gold nanoparticles have a size of 20 to 100 nm.

9. The method of claim 1, wherein the gold nanoparticle-steroid complex has a hydrodynamic size of 25 to 150 nm.

10. The method of claim 1, wherein the gold nanoparticle-steroid complex reduces the expression of pro-inflammatory cytokines and increases the expression of anti-inflammatory cytokines.

11. The method of claim 10, wherein the pro-inflammatory cytokine is TNF-α, IL-1β, IL-6, INF-γ, MMP-1 or MMP-3 and the anti-inflammatory cytokine is IL-4, IL-10 or Arg-1.

12. The method of claim 1, wherein the gold nanoparticle-steroid complex induces repolarization from M1 macrophages to M2 macrophages.


SEQUENCE LISTING (36 sequences)

SEQ ID NO: 1 - DNA, 21 bp, synthetic construct
  misc_feature 1..21: note = TNF-alpha F
  source 1..21: mol_type = other DNA
ctcttctccttcctgatcgtg

SEQ ID NO: 2 - DNA, 21 bp, synthetic construct
  misc_feature 1..21: note = TNF-alpha R
  source 1..21: mol_type = other DNA
ccagagggctgattagagaga

SEQ ID NO: 3 - DNA, 19 bp, synthetic construct
  misc_feature 1..19: note = IL-1beta F
  source 1..19: mol_type = other DNA
gctcgccagtgaaatgatg

SEQ ID NO: 4 - DNA, 18 bp, synthetic construct
  misc_feature 1..18: note = IL-1beta R
  source 1..18: mol_type = other DNA
cagagggcagaggtccag

SEQ ID NO: 5 - DNA, 23 bp, synthetic construct
  misc_feature 1..23: note = IL-6 F
  source 1..23: mol_type = other DNA
cctccagaacagatttgagagta

SEQ ID NO: 6 - DNA, 22 bp, synthetic construct
  misc_feature 1..22: note = IL-6 R
  source 1..22: mol_type = other DNA
tcaggggtggttattgcatcta

SEQ ID NO: 7 - DNA, 19 bp, synthetic construct
  misc_feature 1..19: note = IL-4 F
  source 1..19: mol_type = other DNA
atgggtctcacctcccaac

SEQ ID NO: 8 - DNA, 20 bp, synthetic construct
  misc_feature 1..20: note = IL-4 R
  source 1..20: mol_type = other DNA
atatcgcacttgtgtccgtg

SEQ ID NO: 9 - DNA, 22 bp, synthetic construct
  misc_feature 1..22: note = IL-10 F
  source 1..22: mol_type = other DNA
tgcctttaataagctccaagag

SEQ ID NO: 10 - DNA, 22 bp, synthetic construct
  misc_feature 1..22: note = IL-10 R
  source 1..22: mol_type = other DNA
tcttcattgtcatgtaggcttc

SEQ ID NO: 11 - DNA, 20 bp, synthetic construct
  misc_feature 1..20: note = Arg-1 F
  source 1..20: mol_type = other DNA
aggaattggcaaggtgatgg

SEQ ID NO: 12 - DNA, 20 bp, synthetic construct
  misc_feature 1..20: note = Arg-1 R
  source 1..20: mol_type = other DNA
gtgaaagatgggtccagtcc

SEQ ID NO: 13 - DNA, 20 bp, synthetic construct
  misc_feature 1..20: note = iNOS F
  source 1..20: mol_type = other DNA
tctaccaggaggagatgctg

SEQ ID NO: 14 - DNA, 20 bp, synthetic construct
  misc_feature 1..20: note = iNOS R
  source 1..20: mol_type = other DNA
cctgaacatagaccttgggc

SEQ ID NO: 15 - DNA, 20 bp, synthetic construct
  misc_feature 1..20: note = MMP-1 F
  source 1..20: mol_type = other DNA
gagttcctgacgttggtcac

SEQ ID NO: 16 - DNA, 20 bp, synthetic construct
  misc_feature 1..20: note = MMP-1 R
  source 1..20: mol_type = other DNA
acagcatcttttggcaaatc

SEQ ID NO: 17 - DNA, 20 bp, synthetic construct
  misc_feature 1..20: note = MMP-3 F
  source 1..20: mol_type = other DNA
tcccaagcaaatagctgaag

SEQ ID NO: 18 - DNA, 20 bp, synthetic construct
  misc_feature 1..20: note = MMP-3 R
  source 1..20: mol_type = other DNA
catttgggtcaaactccaac

SEQ ID NO: 19 - DNA, 24 bp, synthetic construct
  misc_feature 1..24: note = GAPDH F
  source 1..24: mol_type = other DNA
gtatgacaacgaatttggctacag

SEQ ID NO: 20 - DNA, 24 bp, synthetic construct
  misc_feature 1..24: note = GAPDH R
  source 1..24: mol_type = other DNA
tctctctcttcctcttgtgctctt

SEQ ID NO: 21 - DNA, 20 bp, synthetic construct
  misc_feature 1..20: note = TNF-alpha F
  source 1..20: mol_type = other DNA
tgtctactgaacttcggggt

SEQ ID NO: 22 - DNA, 19 bp, synthetic construct
  misc_feature 1..19: note = TNF-alpha R
  source 1..19: mol_type = other DNA
gagggtctgggccatagaa

SEQ ID NO: 23 - DNA, 22 bp, synthetic construct
  misc_feature 1..22: note = IL-1beta F
  source 1..22: mol_type = other DNA
ctgttctttgaagttgacggac

SEQ ID NO: 24 - DNA, 20 bp, synthetic construct
  misc_feature 1..20: note = IL-1beta R
  source 1..20: mol_type = other DNA
gcgagatttgaagctggatg

SEQ ID NO: 25 - DNA, 22 bp, synthetic construct
  misc_feature 1..22: note = IL-6 F
  source 1..22: mol_type = other DNA
cttcacaagtcggaggcttaat

SEQ ID NO: 26 - DNA, 22 bp, synthetic construct
  misc_feature 1..22: note = IL-6 R
  source 1..22: mol_type = other DNA
agtgcatcatcgttgttcatac

SEQ ID NO: 27 - DNA, 24 bp, synthetic construct
  misc_feature 1..24: note = IL-4 F
  source 1..24: mol_type = other DNA
atcatcggcattttgaacgaggtc

SEQ ID NO: 28 - DNA, 22 bp, synthetic construct
  misc_feature 1..22: note = IL-4 R
  source 1..22: mol_type = other DNA
accttggaagccctacagacga

SEQ ID NO: 29 - DNA, 21 bp, synthetic construct
  misc_feature 1..21: note = IL-10 F
  source 1..21: mol_type = other DNA
gtcatcgatttctcccctgtg

SEQ ID NO: 30 - DNA, 21 bp, synthetic construct
  misc_feature 1..21: note = IL-10 R
  source 1..21: mol_type = other DNA
gtagacaccttggtcttggag

SEQ ID NO: 31 - DNA, 20 bp, synthetic construct
  misc_feature 1..20: note = Arg-1 F
  source 1..20: mol_type = other DNA
gcagaggtccagaagaatgg

SEQ ID NO: 32 - DNA, 20 bp, synthetic construct
  misc_feature 1..20: note = Arg-1 R
  source 1..20: mol_type = other DNA
acacataggtcagggtggac

SEQ ID NO: 33 - DNA, 20 bp, synthetic construct
  misc_feature 1..20: note = iNOS F
  source 1..20: mol_type = other DNA
gggagccacagcaatatagg

SEQ ID NO: 34 - DNA, 20 bp, synthetic construct
  misc_feature 1..20: note = iNOS R
  source 1..20: mol_type = other DNA
tcagcctcatggtaaacacg

SEQ ID NO: 35 - DNA, 20 bp, synthetic construct
  misc_feature 1..20: note = GAPDH F
  source 1..20: mol_type = other DNA
tgctgagtatgtcgtggagt

SEQ ID NO: 36 - DNA, 21 bp, synthetic construct
  misc_feature 1..21: note = GAPDH R
  source 1..21: mol_type = other DNA
agatgatgacccttttggctc